(19) United States
(12) Patent Application Publication
(10) Pub. No.: US 2023/0029554 A1
(43) Pub. Date: Feb. 2, 2023

(54) ORGANOID CULTURES

(71) Applicant: THE WALTER AND ELIZA HALL INSTITUTE OF MEDECAL RESEARCH, Melbourne (AU)

(72) Inventors: Jordan CLARKE, Melbourne (AU); Yumiko HIROKAWA, Melbourne (AU); MICHELLE PALMIERI, Melbourne (AU); Oliver SIEBER, Melbourne (AU)

(21) Appl. No.: 17/784,159

(22) PCT Filed: Dec. 11, 2020
(86) PCT No.: PCT/AU20/51362

§ 371 Date: Jun. 10, 2022

(30) Foreign Application Priority Data
Dec. 12, 2019 (AU) 2019904705

(51) Int. Cl. C12N 5/09

(52) U.S. Cl. CPC C12N 5/0693; C12N 2533/90; C12N 2513/00; C12N 2527/00; C12N 2501/727

(57) ABSTRACT

The present invention provides improved methods and media for culturing stem cells and propagating organoids. In particular, the methods provide improved methods for culturing normal and tumour or cancer stem cells and their propagation into organoids. Specifically provided are suspension methods with particular utility for scalable organoid expansion, biobanking, experimental manipulation, miniaturization and imaging based high-throughput drug screening.

### INCORPORATION BY REFERENCE

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

The present application claims priority from AU2019904705 filed 12 Dec. 2019, the entire contents of which are herein incorporated by reference.

### BACKGROUND

Cancer remains a major world-wide health burden. Many approaches to cancer treatment have focussed on early detection strategies and prevention. Other approaches have focussed on the development of new and more targeted therapies such as immunotherapies which have resulted in major successes in recent years. A significant challenge in the development of cancer therapies has been the translation of findings from bench to patient, often attributed to the fact that traditional cancer models such as cell lines poorly represent patient tumours, resulting in many drug candidates ultimately failing in clinical trials (Caponigro, G. & Sellers, (2011) Nat. Rev. Drug Discov. 10, 179-187). Although animal cancer models have provided important insights into the biology and treatment of cancer, these only partly capture the clinicopathologic complexity and genetic heterogeneity of human cancers (Cheon, D. J. & Orsulic, S. (2011) Annu. Rev. Pathol. 6, 95-119).

Commonly used human cancer models include cancer cell lines and primary patient-derived tumour xenografts (PDTXs). Cancer cell lines derived from primary patient tissues have contributed substantially to cancer research. However, their generation from primary patient material is generally inefficient and involves extensive tumour cell adaptation and selection to in vitro 2D culture conditions. Additionally, commonly used cancer cell lines (following many passages) often have undergone substantial genetic changes and thus no longer represent the original tumour. PDTX models maintain 3D growth in their animal host and tumour-stroma interactions, but have long establishment and propagation times and are expensive to maintain.

Recent developments in three-dimensional tissue culture techniques and advanced growth factor supplementation have enabled the establishment of patient-derived organoid models for a wide range of human normal and tumour tissues (Drost, J. and H. Clevers, (2018) Nat Rev Cancer 18(7): p. 407-418). Upon embedding into a 3D matrix, tissue-derived adult stem cell cells can be grown with high efficiencies into self-organising organotypic structures, termed organoids. In 2009, Sato et al. (Sato, T., et al., (2009) Nature, 2009. 459(7244): p. 262-5) demonstrated that 3D epithelial intestinal organoids can be established from a single leucine-rich repeat containing G protein-coupled receptor 5 (LGR5)+ intestinal stem cell. Upon embedding into a matrix gel, cells are cultured under serum-free conditions mimicking the in vivo stem cell niche involving WNT3A, R-spondin 1 (a ligand of LGR5), epidermal growth factor (EGF) and bone morphogenic protein (BMP) inhibitor noggin. Consequently, LGR5+ intestinal stem cells grow out as organotypic, highly polarized epithelial structures with proliferative crypt and (for the small intestine) differentiated villus compartments. This culture protocol formed the starting point for other organoid culture protocols of multiple mouse and human tissues.

Organoids can now be routinely generated from fresh, viable tissue samples for many healthy or diseased human organs, representing the organ- or disease-specific cell types derived from stem cells or progenitors (Yip, H. Y. K., et al., (2018) PLoS One, 13(6): p. e0199412). Advantageously, organoids can be readily expanded and passaged, can be cryopreserved and genetically modified, and remain genetically and phenotypically stable over short- to medium-term culture, thus allowing for a wide range of applications in cancer research.

Self-renewal and differentiation of stem cells is governed by growth factors and extracellular matrices (ECM) that provide the required scaffold to support cell attachment and growth during organoid formation. Commonly used scaffolds to support expansion of organoid cultures include basement membrane matrix purified from Engelbreth-Holm-Swarm (EHS) tumour, for example, Matrigel®, Geltrex® or Cultrex® matrix. Alternatively, synthetic or semi-synthetic hydrogels may be used. These matrices form a solid gel at 37° C. (typically the temperature at which organoids are cultured).

Organoid models are faster, easier, and less expensive to generate than patient-derived xenograft (PDX) mouse models. Organoids can in principal capture all stages of tumourigenesis and cancer genomic subtypes, and they can be used to uncover biological changes underlying metastatic progression. Importantly, organoid cultures can be subjected to a wide range of functional assays, and they can be rapidly tested for their sensitivity to novel drugs, drug combinations, and drugs given in defined temporal sequences. Organoid model systems can be expanded to contain stromal or immune cells, with the latter particularly pertinent to the development and in vitro testing of new immunotherapies (Pauli, C., et al., (2017) Cancer Discov, 7(5): p. 462-477; Drost, J. and H. Clevers, (2018) Nat Rev Cancer, 18(7): p. 407-418). Organoids also have applications in the study of organogenesis, disease modelling, toxicity assays, and regenerative medicine. For example, genome editing of organoid cultures can allow evaluation of patient-specific mutations found in certain cancers. Other applications include organoid co-culture with pathogenic bacteria or viruses to study infections, the use of organoids to propagate intracellular pathogens, or to produce biological products.

Organoid culture conditions for human intestinal tissues (and mouse intestinal tissues), both normal and malignant, have been reported (Sato, T., et al., (2009) Nature, 459(7244): p. 262-5; Sato, T. and H. Clevers, (2013) Science, 2013. 340(6137): p. 1190-4). Briefly, isolated normal intestinal crypts or tumour fragments are embedded or grown on top of a solid biological matrix similar to an endogenous basement membrane (such as Matrigel®, Geltrex® or Cultrex® matrix) or a synthetic or semi-synthetic hydrogel to recapitulate “mini-guts” of “mini-tumours”. Cultures of normal tissue are supported by a cocktail of growth factors to activate the signalling pathways necessary for the renewal of stem cells. This includes stimulation of WNT and EGF signalling (with WNT3A, R-spondin and EGF) and inhibition of BMP, TGF-beta and p38 signalling (with noggin, A83-01 and SB202190). Over several days, cell clusters grow into mature and self-organizing organoids, containing polarised cells and representative epithelial architecture. For growth of organoids from tumour tissue, key growth factors such as WNT3A are omitted from the media as colorectal cancers have mutations that constitutively activate this pathway. This approach permits selective growth of tumour cells as normal cells cannot grow in media that lack WNT. Organoid cultures can reproducibly be derived from human colorectal normal epithelium and adenocarcinoma tissues (Sato, T., et al., (2011) Gastroenterology 141(5): p. 1762-72). Furthermore, recent studies have confirmed that cancer organoid cultures maintain primary tumour molecular profiles during short- to medium-term culture when examined at the genetic, transcriptional and proteomic levels, and combined with drug sensitivity testing, can facilitate elucidation of molecular mechanisms of drug resistance (Cristobal, A., et al., (2017) Cell Rep, 2017. 18(1): p. 263-274). Additionally, organoid drug testing in vitro has been shown to hold promise for guiding the delivery of drug treatments in initial proof-of-concept patient cohort studies (Ooft, S. N., et al., (2019) Sci Transl Med, 2019. 11(513)).

While cell embedment into a solid support matrix is considered essential for intestinal epithelial organoid culture, this presents technical challenges that impede organoid expansion, experimentation, and high-throughput screening applications. Organoid growth in solid matrices is constrained due to solid stress accumulation, oxygen and nutrient delivery requiring frequent passaging to maintain and expand cultures. Solid matrices must be mechanically or enzymatically removed to isolate organoids for propagation or experimental applications. There is need in the art for improved methods of generating and propagating organoid cultures.

### SUMMARY OF THE DISCLOSURE

The present invention is premised on the finding that a low viscosity solubilised basement membrane preparation provides particular advantages for culturing organoids compared to solid-state basement membrane preparations (such as Matrigel®, Geltrex® or Cultrex® matrix) or hydrogels traditionally used for the growth of normal and tumour organoids. The 3-D matrix produced by solid-state basement membrane preparations or hydrogels is considered a key requirement for the growth and propagation of normal and tumour organoids. Basement membrane matrices such as Matrigel®, Geltrex® or Cultrex® are a liquid at 4° C., but form a solid gel at 37° C. Synthetic or semi-synthetic hydrogels require the addition of a cross-linker to solidify. Disadvantages of using such traditional matrices, however, include that they are challenging to use in high-throughput liquid handling applications due to the cooling requirement to avoid matrix setting during dispensing (for basement membrane preparations), require the extraction of embedded organoids for propagation limiting large-scale expansion, and are often associated with high costs for the matrix reagents.

The present inventors have developed a low-viscosity matrix suspension (LVM) culture method that enables growth of normal and cancer organoids from the human large intestine. The suspension culture has particular utility for scalable organoid expansion, biobanking, experimental manipulation, miniaturization and imaging based high-throughput drug screening.

Normal and tumour organoids can be cultured in a low percentage, liquid-state basement membrane preparation without the need for a solid basement membrane or hydrogel matrix as a support. The method is scalable from 1,536-well plates to large-volume bioreactors. The method is therefore particularly useful for high throughput organoid workflows such as generation, propagation, large-scale expansion and drug screening of individual patient organoids.

The present disclosure provides a method for culturing stem cells, the method comprising:

(i) providing a low viscosity extracellular matrix;

(ii) culturing the stem cells in a stem cell culture medium comprising the extracellular matrix and a cell culture support medium.

In one example, the stem cells are cultured in suspension culture.

In one example, the stem cells are capable of forming organoids when cultured.

In one example, the low viscosity extracellular matrix is a basement membrane protein mixture extracted from Engelbreth-Holm-Swarm (EHS) mouse sarcoma.

In another example, the low viscosity extracellular matrix is a natural, synthetic or semi-synthetic hydrogel at a concentration below which it solidifies.

The term “low viscosity” as used herein is intended to refer to a matrix solution which remains in liquid form at a temperature in which stem cells are normally cultured, preferably a temperature between about 35° C. and 39° C., preferably 37° C. In a particular example, the low viscosity extracellular matrix is a 3-40% v/v matrix, more particularly, a 3-10% v/v matrix, even more particularly a 3-5% v/v matrix.

In some examples the extracellular matrix comprises laminin, collagen IV, enactin and/or proteoglycans.

In some examples, the extracellular matrix is a low viscosity matrix suspension provided at 3-5% of the final culture media volume. In other examples, the final concentration of extracellular matrix is about 200-500 μg/ml.

In some examples, the method according to the present disclosure comprises providing a low viscosity extracellular matrix solution, the matrix comprising about 60% laminin, 30% collagen IV, 8% enactin and 2% proteoglycans. The extracellular matrix is preferably provided as a solution wherein the matrix is diluted to the appropriate concentration in a cell culture medium or in the cell culture support medium described herein.

In some examples, the cell culture support medium is a cancer organoid medium (reduced medium) or “complete” medium (for normal organoids) as described herein.

“Reduced medium” as referred to herein means a medium comprising DMEM/F12, HEPES, B27 supplement, N2 supplement, nicotinamide, N-acetyl cysteine, EGF, penicillin-streptomycin and bFGF.

In some examples, the cell culture support medium comprises a liquid basal medium. The term cell culture support medium is synonymous with medium, culture medium or cell medium. In some examples the basal medium may be selected from the group consisting of DMEM/F12, Advanced RPMI or RPMI 1640.

In some examples, the extracellular matrix may be supplemented with one or more growth factors. In some examples, the one or more growth factors are selected from the group consisting of TGF-beta, epidermal growth factor, insulin-like growth factor, fibroblast growth factor, tissue plasminogen activator and other growth factors which occur naturally in the EHS tumour.

In a particular example, the method comprises providing a low viscosity extracellular matrix as described herein supplemented with Epidermal Growth Factor (EGF) at a concentration range of about 0.5-1.3 ng/ml; Basic Fibroblast Growth Factor (bFGF) at a concentration range of about <0.1-0.2 pg/ml; Nerve Growth Factor (NGF) at a concentration range of about <0.2 ng/ml; Platelet Derived Growth Factor (PDGF) at a concentration range of about 5-48 pg/ml; Insulin-like Growth Factor-1 (IGF-1) at a concentration range of about 11-24 ng/ml; and Transforming Growth Factor-beta (TGF-δ) at a concentration range of about 1.7-4.7 ng/ml.

In some examples, the liquid basal medium is supplemented with one or more antibiotics or anti-fungal agents. In one example, the antibiotics are selected from the group consisting of gentamycin, kanamycin, amphotericin, penicillin and streptomycin. In a particular example, the culture medium is supplemented with gentamycin, kanamycin, amphotericin and penicillin-streptomycin. In another example, the basal medium comprises an anti-fungal. In one example, the anti-fungal is nystatin. In one example, the antibiotics are primocin or normocin.

In some examples, the liquid basal medium is supplemented with one or more growth factors as well as nicotinamide, N-acetyl-L-cysteine, B27 supplement, N2 supplement and HEPES buffer. In one example, the growth factors are selected from the group consisting of Basic Fibroblast Growth Factor (bFGF) and Epidermal Growth Factor (EGF). Preferred concentrations are 0.01M nicotinamide, 1 mM N-acetyl-L-cysteine, 1×B27 supplement, 1×N2 supplement and 100 mM HEPES buffer. In one example, the growth factors are selected from the group consisting of 20 ng/mL Basic Fibroblast Growth Factor (bFGF) and 50 ng/mL Epidermal Growth Factor (EGF).

In a further example, the liquid basal medium comprises one or more of Wnt3A protein, spondin, nystatin and noggin.

In a further example, the method according to the disclosure comprises a liquid basal medium comprising a ROCK1 inhibitor. The addition of a ROCK1 inhibitor has been found to prevent anoikis, especially when culturing single stem cells. The ROCK1 inhibitor is preferably selected from R-(+)-trans-4-(1-aminoethyl)-N-(4-Pyridyl)cyclohexanecarboxamide dihydrochloride monohydrate (Y-27632; Sigma-Aldrich), 5-(1,4-diazepan-1-ylsulfonyl)isoquinoline (fasudil or HA1077; Cayman Chemical), and (S)-(+)-2-methyl-1-[(4-methyl-5-isoquinolinyl)sulfonyl]-hexahydro-1H-1,4-diazepine dihydrochloride (H-1152; Tocris Bioscience). In one example, the ROCK1 inhibitor, for example Y-27632, is added to the culture medium only on the day of initial cell, tissue fragment or organoid seeding. A concentration for Y27632 may be between 0.01 to 100 μM, preferably 10 μM.

Preferably, the ROCK1 inhibitor is Y27632.

In another example, the basal medium comprises Dulbecco's Modified Eagle Medium Nutrient Mixture F12 (DMEM/F12) or Advanced DMEM/F12. The skilled person will appreciate that other commercial culture mediums may be supplemented for DMEM/F12 as necessary.

In a particular example, the method comprises culturing the stem cells in a stem cell culture support medium (e.g. liquid basal medium) comprising about 3-10%, more preferably 3-5% low viscosity extracellular matrix

In a preferred example, the extracellular matrix is a dilution of a “traditional basement membrane matrix” such, for example, Matrigel®, Geltrex® or Cultrex® matrix. By “traditional basement membrane matrix” it is meant a preparation comprising about 60% laminin, 30% collagen IV, 8% enactin and 2% proteoglycans which adopts a gel consistency at 22° C. to 37° C.

In the present disclosure, a traditional basement membrane matrix as described herein is diluted to about 3-40% v/v, preferably about 3-10% v/v, more preferably about 3-5% v/v. In some examples, the traditional basement membrane matrix is diluted in the cell culture support medium described herein.

In a preferred example, the method comprises culturing the stem cells in a stem cell culture medium comprising:

(i) a low viscosity extracellular matrix as described herein; and

(ii) a cell culture support medium.

In one example, the cell culture support medium comprises a liquid basal media as described herein.

In one example, the liquid basal media comprises at least one antibiotic, at least one anti-fungal agent and a ROCK1 inhibitor. In one example, the ROCK1 inhibitor is provided on the day of initial cell, tissue fragment or organoid seeding.

In another example, the liquid basal media comprises one or more of gentamycin, kanamycin, amphotericin, penicillin/streptomycin, nystatin, and a ROCK1 inhibitor.

In another example, the liquid basal media comprises primocin or normocin and penicillin/streptomycin.

In one example, the extracellular matrix is a basement membrane protein mixture extracted from Engelbreth-Holm-Swarm (EHS) mouse sarcoma. In another example, the low viscosity extracellular matrix is a 3-10% dilution of Matrigel®, Geltrex® or Cultrex® (BME type I or II), or collagen type I-A. In a further example, the Matrigel®, Geltrex® or Cultrex® (BME type I or II) or collagen type I-A is diluted in cell culture medium.

In a further example, the liquid basal media is supplemented with one or more growth factors as well as one or more of nicotinamide, N-acetyl-L-cysteine, B27 supplement, N2 supplement and HEPES buffer.

In some examples, the methods further comprise seeding the stem cell culture medium with cells, preferably stem cells. In some examples, the cells are of a type capable of forming organoids.

The methods of the disclosure allows culturing of normal stem cells, isolated fragments from the small intestine, colon, rectum, skin, breast, oesophagus, head and neck, liver, lung, prostate, kidney, bladder, thyroid, endometrial, stomach and pancreas comprising the stem cells while preserving the presence of stem cells that retain an undifferentiated phenotype and self-maintenance capabilities. For example, isolated crypts derived from normal colon epithelium that are cultured according to a method of the disclosure develop into crypt organoids comprising a central lumen lined by crypt-like protrusions.

The stem cells according to the methods described herein may be embryonic stem cells (ESC), mesenchymal stem cells (MSC), induced pluripotent stem cells (iPSC) or adult stem cells.

The method of the disclosure also allows for the culturing of normal and tumour or cancer stem cells (CSCs) and their propagation into organoids.

In some examples, the method provides for the culture of stem cells derived from primary tumours from the small intestine, colon, rectum, skin, breast, oesophagus, head and neck, liver, lung, prostate, kidney, bladder, thyroid, endometrial, stomach and pancreas. In some examples, the method provides for the culture of stem cells derived from metastatic tumours from the small intestine, colon, rectum, skin, breast, oesophagus, head and neck, liver, lung, prostate, kidney, bladder, thyroid, endometrial, stomach and pancreas. In some examples, the stem cells are present in a cancer biopsy sample. In another example the stem cells are circulating tumour cells retrieved from blood samples.

In some examples, the normal tissue biopsy sample is subjected to a digestion step to tissue fragments of less than 5 mm or single cells prior to being added to the extracellular matrix. In another example, the primary or metastatic tumour tissue biopsy sample is subjected to a digestion step to tissue fragments of less than 5 mm or single cells prior to being added to the extracellular matrix.

In some examples, the normal or tumour tissue fragments are resuspended at a concentration of 50 to 20,000 fragments per ml of liquid basal media with low viscosity basement membrane matrix. In another example the single normal or tumour cells are resuspended at a concentration of 200-2,000,000 cells per ml of liquid basal media with low viscosity basement membrane matrix.

In a further example, the tissue fragments or cells are cultured for a period of time sufficient to generate organoids. In one example, the culture period is between two and 100 days, between 2 and 70 days, between 2 and 50 days, between 2 and 20 days or between 10 and 20 days.

In a particular example, the methods further comprise agitating the tissue fragments or cells during culture. In some examples, the agitation is performed when establishing the culture from tissue fragments. In some examples, the agitation is performed during maintenance of established cultures. In some examples, agitation is performed during a media change. Alternatively, the cells may be agitated at a frequency determined by the skilled person. For example the tissue or cells may be agitated every few days, weekly, twice weekly, or once or month as necessary to avoid aggregation. Methods of agitation will be familiar to persons skilled in the art. Examples include pipetting with a pipette. In another example the tissue or cells may be agitated using a shaker or vortex mixer.

The inventors have found that cells or organoids tend to precipitate to the bottom of the culture dish with gravity even in the presence of 3-5% Matrigel/basement membrane. Organoids of epithelial origin grow best when attached to the Matrigel/basement membrane but will stop growing when adhered to a plastic or glass surface of the culture dish.

Accordingly, the methods of the disclosure also comprise the additional step of periodically agitating the cells in the stem cell culture medium.

The disclosure also provides a method of producing organoids, the method comprising:

(i) providing a stem cell culture medium comprising low viscosity extracellular matrix and a cell culture support medium;

(ii) seeding the stem cell culture medium with stem cells; and

(iii) culturing the cells in the medium under a time and conditions sufficient for the cells to generate organoids.

In one example, the cells are seeded at about 10,000-100,000 cells/mL

In another example, the cells are cultured for between 2 and 50 days, between 2 and 20 days or between 10 and 20 days.

In a further example, the cells are cultured according to the methods described herein at a temperature between 35° C. and 39° C., preferably about 37° C.

In a further example, the cells are cultured in 5-10% CO₂.

The disclosure also provides a stem cell culture medium for culturing stem cells, comprising:

(i) a low viscosity extracellular matrix as described herein; and

(ii) a cell culture support medium.

In one example, the cell support media comprises a liquid basal medium as described herein. In one example, the liquid basal medium comprising at least one antibiotic, at least one anti-fungal agent and a ROCK1 inhibitor.

In one example, the liquid basal medium/stem cell support medium comprises one or more of gentamycin, kanamycin, amphotericin, penicillin/streptomycin, nystatin, primocin, normocin and a ROCK1 inhibitor.

In one example, the extracellular matrix is a basement membrane protein mixture extracted from Engelbreth-Holm-Swarm (EHS) mouse sarcoma. In another example, the low viscosity extracellular matrix is a 3-10% dilution of Matrigel®, Geltrex® or Cultrex®. In a further example, the Matrigel®, Geltrex® or Cultrex® is diluted in the cell culture support medium.

In a further example, the liquid basal media is supplemented with one or more growth factors as well as one or more of nicotinamide, N-acetyl-L-cysteine, B27 supplement, N2 supplement and HEPES buffer.

In a further example, the liquid basal medium/stem cell support medium comprises one or more of gentamycin, kanamycin, amphotericin, penicillin/streptomycin, primocin, normocin nystatin, and a ROCK1 inhibitor.

In a further example, the stem cell support medium comprises one or more of DMEM/F12, HEPES, B27 supplement, N2 supplement, nicotinamide, N-acetyl-L-cysteine, Wnt3A, R-spondin-2, EGF, noggin, A83-01 (TGFβ kinase/active receptor-like kinase (ALK 5) inhibitor), SB202190 (p38 MAP kinase inhibitor), penicillin-streptomycin normocin or primocin.

The disclosure also provides use of the stem cell culture medium as described herein for culturing stem cells or isolated organoid structures comprising those stem cells.

In one example, the cells are adult stem cells, more particularly normal or cancer stem cells or tissue fragments comprising the same. The source of the cells may be derived from small or large intestine, colon, rectum, skin, breast, oesophagus, head and neck, liver, lung, prostate, kidney, bladder, thyroid, endometrial, stomach and pancreas.

The disclosure further provides the use of the stem cell culture medium according to the disclosure for culturing cancer stem cells or tumour fragments comprising cancer stem cells that form organoids. The source of the cells may be derived from small or large intestine, colon, rectum, skin, breast, oesophagus, head and neck, liver, lung, prostate, kidney, bladder, thyroid, endometrial, stomach and pancreas.

The disclosure also provides crypt-organoids from intestinal epithelium, comprising a central lumen lined by crypt-like protrusions that result from culturing of normal epithelial stem cells or isolated crypts in a culture medium of the disclosure. In one example, the crypt organoid is obtainable using a method of the disclosure.

The disclosure also provides the use of normal or tumour organoids produced by the methods described herein in one or more applications selected from the group consisting of:

(i) a drug discovery screen;
(ii) large-scale organoid production;
(iii) functional assay;
(iv) genetic testing assay;
(v) drug testing assay;
(vi) infection assay;
(vii) pathogen expansion assay;
(viii) co-culture assay;
(ix) transplantation assay;
(x), gene editing assay;
(xi) toxicity assay;
(xii) the production of biological products; and
(xiii) regenerative medicine.

The disclosure also provides a population of stem cells or organoids comprising the stem cells obtained using the stem cell culture medium of the disclosure.

The disclosure also provides a population of organoids produced by culturing stem cells according to a method of the disclosure.

### DESCRIPTION OF THE FIGURES

FIG. 1 shows representative brightfield images and quantification of (A) colorectal cancer organoids and (B) normal colorectal epithelial organoids grown embedded in solid-state Matrigel matrix or in suspension in low-viscosity 5% Matrigel matrix. Organoids were disaggregated and cell numbers were quantified at 14 days post seeding of single cells. Quantified data are for mean+/−SEM for three biological replicates. Scale bar represents 100 μm.

FIG. 2 shows drug sensitivity testing of colorectal cancer organoids in low-viscosity 3-5% Matrigel matrix. (A) Organoids were established from single cells in triplicate in 384-well plates and imaged 6 days after addition of 5-fluorouracil (5-FU) and SN38 (active metabolite of irinotecan). (B) Organoid growth at the population level for triplicate wells as quantified from bright field images over the 6-day time course. (C) Organoid growth at the individual level for a representative well as quantified from bright field images over the 6-day time course. (D) ED50 determination from bright field images by image analysis at 6 days post addition of 5-FU and SN38. Quantified data are for mean+/−SEM for >350 organoids.

FIG. 3 shows dose response curves and ED50 determination from CellTiter Glo® viability assays for a pro-apoptotic drug combination conducted in colorectal cancer organoids embedded in solid-state Matrigel or in suspension in low-viscosity 3-5% Matrigel matrix at 1 day post drug addition. Quantified data are for mean+/−SEM for triplicate wells.

FIG. 4 shows representative images of large-scale expansion cultures for normal epithelium and colorectal cancer organoids grown in Bioreactor Tubes. A total of 3.5 ml of both Normal or Cancer Organoid Media comprising low-viscosity 3-5% Matrigel matrix containing 100,000 cells was cultures in a well of 6 well plate. A total of 7 ml of cell suspension containing 200,000 cells was cultured in 50 ml Mini Bioreactor Tubes (Corning) for 14 days. Scale bar represents 100 μm.

FIG. 5 shows examples of organoid cultures from human breast cancer. A total of 3.5 mL of Cancer Organoid Media comprising low-viscosity 3-5% Matrigel matrix containing 100,000 cells was cultured in a well of a 6 well-plate. Organoids were grown over 14 days. Scale bar represents 100 μm.

FIG. 6 shows a-b, Comparison of (a) establishment rates (passage 0 to 1) and (b) times (passage 0 to 1) in donut culture between cancer organoids grown in complete or reduced medium. c-d, Comparison of (c) propagation rates (passage 1 to 2) and (d) times (passage 1 to 2) in dome culture between cancer organoids grown in complete or reduced medium. Data (b, d) are plotted as mean±s.d. Statistical significance (b, d) was attributed to values of p<0.05 as determined by the Student's t test. NS, p>0.05.

FIG. 7 shows low-viscosity matrix suspension culture for propagation of organoids from normal colorectal epithelium and cancer. a-b, Representative images of two independent normal colorectal organoids grown in (a) Matrigel dome culture and (b) low-viscosity matrix suspension culture. c-d, Representative images of two independent cancer organoids grown in (c) Matrigel dome culture and (d) low-viscosity matrix suspension culture; low-magnification images, scale bar, 500 μm; high-magnification images, scale bar, 200 μm.

FIG. 8 Low-viscosity matrix suspension culture for propagation of organoids from normal colorectal epithelium and cancer. a-b, Comparison of propagation rates and times (passage 1 to 2) between LVM suspension and dome culture methods for normal and cancer organoids. c-e, Comparison of propagation times for normal and cancer organoids grown in LVM suspension or dome culture according to location and tumor stage. f-g, Comparison of live cell yield per μl of Matrigel for normal and cancer organoids grown for 14 days in LVM suspension or dome culture; scale bar, 200 μm. Data (b, f, g) are plotted as mean±s.d. Statistical significance (b, f, g) was attributed to values of P<0.05 as determined by the Student's t test. NS, P>0.05, *P<0.05, **P<0.01, ***P<0.001.

FIG. 9 shows low-viscosity matrix suspension cultures of both normal colorectal and cancer organoids using different types of support matrices. Normal and cancer organoids from patients were grown for 14 days as suspension cultures in 5% Matrigel, BME-1, BME-2 or collagen type 1A; low-magnification images, scale bar, 500 μm; high-magnification images, scale bar, 200 μm.

FIG. 10 shows Low-viscosity suspension culture conditions support three-dimensional growth of human cancer cell lines. a—Representative images from human cancer cell lines from prostate (PC-3), breast (MCF-7, MDA-MB-231) and pancreas (BxPC-3) grown for 7 to 10 days in classic 2-D conditions and LVM suspension conditions; scale bars, 100 μm.

FIG. 11 shows normal colorectal and cancer organoids grown in low-viscosity matrix suspension recapitulate morphological development observed in dome culture. a-b, Representative brightfield images of normal and cancer organoids grown in either LVM suspension or dome culture over a 14-day period; scale bar, 200 μm. c-d, Immunofluorescence microscopy images of normal and cancer organoids grown in LVM suspension stained with (c) Ki-67 and (d) E-cadherin; scale bar, 100 μm for Ki-67, 50 μm for E-cadherin. e-f, Representative hematoxylin and eosin stained sections of primary tissues and organoids for (e) normal and (f) cancer specimens; scale bar, 200 μm.

FIG. 12 shows colorectal cancer organoids grown in low-viscosity matrix suspension culture maintain histopathological similarities as compared to the original primary tissues. Representative hematoxylin and eosin stained sections of primary cancers tissues and organoids for 10 patients; scale bar, 200 μm.

FIG. 13 shows global genomic alterations in human colorectal cancer organoids. (a), Mutation profiles in 26 colorectal cancer organoids and 224 TOGA colorectal cancers. Counts of SNVs and InDels, and proportions of nucleotide transitions and transversions are reported, split into distinct hypermutated and non-hypermutated cases. (b), Mutation frequencies of major colorectal cancer driver genes for cancer organoids and TOGA-analyzed cancers. (c), Proportions of samples with relative DNA copy number alterations for cancer organoid and TOGA-analyzed cancers. (d), Genome-wide DNA copy number aberrations for cancer organoids stratified into non-hypermutated and hypermutated (MSI-H) cases.

FIG. 14 shows scalable expansion of intestinal normal and cancer organoids in bioreactor tubes. a-b, Comparison of (a) establishment rates and (b) times (passage 0 to 1) between LVM suspension and donut culture methods for normal (n=31 and n=91, respectively) and cancer organoids (n=16 and n=75, respectively). c-e, Comparison of establishment times for normal and cancer organoids grown in LVM suspension or donut culture according to (c, d) location and (e) tumor stage. f, Quantification of normal and cancer organoid growth in bioreactor tubes. A total of 200,000 cells were seeded in 7 ml LVM suspension culture medium per tube; two independent normal and three independent cancer organoid cultures were assayed in duplicate. g, Representative images of normal and cancer organoids grown in bioreactor tubes for 4-8 weeks, resulting in substantial enlargement of organoids and/or organoid aggregates; scale bars, 200 μm. Data (b) are plotted as mean±s.d. Statistical significance (b) was attributed to values of p<0.05 as determined by the Student's t test. NS, p>0.05, ***p<0.001.

FIG. 15 shows plate uniformity of cancer organoid viability assays with low-viscosity matrix suspension culture of intestinal organoids in 384-well format. (a), Schematic of the workflow for organoid viability assessment. Established organoids were dissociated and seeded as single cells, grown into small organoids over 3 days and treated with drug for 7 days with daily bright-field z-stack imaging. Cell viability was determined by both image analysis and CellTitreGlo 3D assays. (b), Representative images of vehicle treated organoids in 384-well format on day 3 and day 10 of the assay, scale bars, 100 μm. c-d, Three independent cancer organoids were examined for uniformity of maximum (Max) and minimum (Min) signals; (c) plate signals were visualized by heat maps, and (d) raw signals of mean organoid size or relative luminescence units (RLU) were plotted against the respective plate column. e-f, Three cancer organoids were examined for uniformity of drug dose-response curves; (e) plate signals were visualized by heat maps, and (f) four-parameter logistic regression was used to fit drug dose-response curves for each side of each plate (left and right); each color represents a different plate row.

FIG. 16 shows low-viscosity matrix suspension culture of intestinal organoids in 384-well format. (a), Overview of the image processing pipeline for determination of organoid sizes, including flattening of bright-field z-stack images, removal of background and identification of organoids. (b), Assessment of plate edge effects for vehicle treated organoids after 10 days in culture. Mean organoid size and CellTitreGlo 3D luminescence signals were used for generating plate heatmaps; areas used for drug screen assays are marked with black boxes. Data are plotted as for each column as mean±s.e.m.

FIG. 17 shows inhibition of colorectal cancer organoid growth using the proteasome inhibitor bortezomib. a-b, Three independent cancer organoids were assayed in triplicate drug titrations and four-parameter logistic regression was used to fit drug dose-response curves to (a) mean organoid size measurements by image analysis and (b) relative luminescence units (RLU) as determined by CellTitreGlo 3D assays. The bortezomib concentration of 1 um was selected as positive (killing) control, indicated by the dashed red line.

FIG. 18 shows reproducibility of imaging-based drug sensitivity testing of cancer organoids for clinically relevant agents including 5-fluorouracil (5-FU), oxaliplatin, SN-38, regorafenib and TAS-102. (a), Three independent organoids were assayed in two independent runs consisting of duplicate drug titrations (corresponding 200-400 organoids) for 5-FU, oxaliplatin, SN-38, regorafenib and TAS-102. Organoid sizes were calculated across duplicate wells and plotted as mean±s.e.m. Four-parameter logistic regression was used to fit drug dose-response curves. b-d, Between run correlations for (b) pGR50 (c) GRmax and (d) GRaoc estimates. Statistical significance (b-d) was attributed to values of p<0.05 as determined by the t-test and Pearson's correlation coefficient (r).

### DETAILED DESCRIPTION OF THE INVENTION

### General Techniques and Selected Definitions

The term “and/or”, e.g., “X and/or Y” shall be understood to mean either “X and Y” or “X or Y” and shall be taken to provide explicit support for both meanings or for either meaning.

As used herein, the terms “a”, “an” and “the” include both singular and plural aspects, unless the context clearly indicates otherwise.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example described herein is to be applied mutatis mutandis to each and every other example of the disclosure unless specifically stated otherwise.

Those skilled in the art will appreciate that the disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Unless specifically defined otherwise, all technical and scientific terms used herein shall be taken to have the same meaning as commonly understood by one of ordinary skill in the art (for example, in cell culture, immunohistochemistry, protein chemistry, and biochemistry).

The term “about”, as used herein when referring to a measurable value such as an amount of weight, time, dose, etc. is meant to encompass variations of ±20% or ±10%, more preferably ±5%, even more preferably ±1%, and still more preferably ±0.1% from the specified amount, as such variations are appropriate to perform the disclosed method.

The term “organoid” as used herein refers to tiny, self-organised three-dimensional tissue cultures derived from stem cells. In one example, the organoid is derived from colon or rectum and exhibits crypt-like structures surrounding a central lumen filled with apoptotic cell bodies. Organoids can be established from patient-derived healthy and tumour tissue samples and can be molecularly characterised.

The term “extracellular matrix” as used herein refers to a three-dimensional network of basement membrane macromolecules such as collagen, enzymes and glycoproteins that provide structural and biochemical support for surrounding or embedded cells. The term also encompasses a synthetic or semi-synthetic hydrogel.

The term “basement membrane” as used herein refers to a thin, fibrous, extracellular matrix of tissue that separates the lining of an internal or external body surface from the underlying tissue. This surface may be epithelium (skin, respiratory tract, gastrointestinal tract, etc.), mesothelium (pleural cavity, peritoneal cavity, pericardial cavity, etc.) and endothelium (blood vessels, lymph vessels, etc.).

The term “liquid basal medium” refers to a cell culture medium which promotes the growth of cells. Cell culture medium typically contains sugars, amino acids, buffers, salts, vitamins and trace metals.

The term “stem cell culture medium” as used herein refers to a medium comprising a low viscosity extracellular matrix and cell culture support medium as described herein.

The term “cell culture support medium” as used herein refers to a liquid basal medium that is used to support the growth of stem cells into organoid cultures. For example, a typical formulation of support medium may include DMEF/F12, HEPES, B27 supplement, N2 supplement, nicotinamide, N-acetyl-L-cysteine, Wnt3A, R-spondin-2, EGF, noggin, A83-01, SB202190, penicillin-streptomycin, normocin or primocin.

An “inducible pluripotent stem cell (iPS or iPSC)” refers to a type of pluripotent stem cell that can be generated directly from adult cells. iPSCs are typically derived by introducing products of specific sets of pluripotency-associated genes or reprogramming factors (Oct4, Sox2, c-Myc and Klf4) into a given cell type. They can propagate indefinitely, as well as give rise to every other cell type in the body.

An “embryonic stem cell (ESC)” refers to a stem cell derived from the undifferentiated inner mass cells of a human embryo. ESCs are pluripotent and thus able to differentiated into all derivatives of the three primary germ layers (ectoderm, endoderm and mesoderm).

An “adult stem cell (ASC)” refers to an undifferentiated cell found among differentiated cells in a tissue or organ. An adult stem cell can renew itself and can differentiate to yield some of all of the major specialised cell types of the tissue or organ. Hematopoietic stem cells and Mesenchymal stem cells are examples of bone marrow-derived stem cells. Adult stem cells have also been identified, for example, in the brain, peripheral blood, skeletal muscle, blood vessels, skin, heart, gut, liver, ovary and testis.

By “tumour or cancer stem cell” it is meant a cell that possesses characteristics associated with normal stem cells, specifically the ability to give rise to all cell types found in the particular cancer sample. CSCs are tumorigenic and may generate tumours through the stem cell processes of self-renewal and differentiation into multiple cell types. Cancer stem cells can thus only be defined experimentally by their ability to recapitulate the generation of a continuously growing tumour”. Alternative terms in the literature include tumour-initiating cell and tumorigenic cell. The gold-standard assay currently is serial xeno-transplantation into immunodeficient mice.

### Organoids

Organoids are self-organising three-dimensional (3D) in vitro tissue models that recapitulate many of the physiologically relevant features of the normal or diseased tissue from which they are derived. Organoid technology allows the establishment of long-term stem cell-based organotypic cultures in the absence of feeder cells by supplementing the culture medium with well-defined stem cell niche factors. A variety of different organoid culture methods have been described over the past years. Organoids can be derived from either pluripotent stem cells (PSCs), i.e., ESCs and induced PSCs (iPSCs), or multipotent organ-specific adult stem cells (ASCs), and be composed of either only epithelial cells or both epithelial and mesenchymal cell types. The rapid advancement of organoid technology promises exciting new insights into developmental biology and opens new avenues for necessary clinical approaches toward treating human disease and regenerative medicine.

The term “organoid” was initially used in oncology. From the 1960s onward, the term was applied to organotypic cultures that self-organized during cell sorting and reaggregation experiments (Lancaster and Knoblich, (2014) Science 345:124-125). Later, the definition of organoids became refined to three-dimensional (3D) in vitro grown structures derived from PSCs or ASCs that self-organise into a near-native microanatomy with organ-specific differentiated cell types and tissue compartmentalisation.

The identification of leucine-rich repeat containing G-protein-coupled receptor 5 (Lgr5) as intestinal stem cell-specific marker gene allowing for characterization and purification of intestinal stem cells (Barker et al., (2007) Nature 449:1003-1007) and, subsequently, the understanding that adult intestinal stem cells can be both proliferative and long lived in vivo led to the use of isolated Lgr5+ stem cells as a putative cellular source for organoid cultures.

Furthermore, the organoid culture medium containing the niche factors Wnt Family Member 3A (Wnt3A), epidermal growth factor (EGF), Noggin, and R-spondin was defined following the discovery of Wnt signalling as an essential cellular signalling pathway for stem cell maintenance in vivo (Korinek et al., (1998) Nat Genet 19:379-383), the observation that R-spondins, later identified as Wnt agonists binding to LGR5, are mitogens causing stem cell hyperplasia, and the study of the contrasting roles of tyrosine kinase receptor signalling, with EGF being another potent mitogen and bone morphogenetic protein (BMP) inhibitor Noggin being crucial for the maintenance of stem cell niche. In addition, ROCK1 inhibitor was added to the primary cultures to inhibit anoikis that was previously observed in purified colonic epithelial cells and, subsequently, the understanding that adult intestinal stem cells can be both proliferative and long lived in vivo (Barker et al., (2007) Nature 449:1003-1007) led to the use of isolated Lgr5+ stem cells as a putative cellular source for organoid cultures.

Embedding of the purified Lgr5+ stem cells in Matrigel as an ex vivo substitute for the extracellular matrix (ECM) was later developed following previous experience with feeder layer cultures which showed that fibroblasts producing ECM to support stem cell growth and preincubation of tissue culture plastic with ECM proteins such as collagen or laminin further enhanced stem cell clonogenicity (Jensen K B et al. (2010) Nat. Protoc. 5:898-911) and the observation that ECM hydrogels such as Matrigel foster 3D aggregation and polarisation of stem cells (Xu et al. (2001) Nat. Biotechnol. 19:971-974).

In 2009, it was demonstrated (Sato T et al. (2009) Nature 459:262-265) that self-organising near-native intestinal epithelial structures can be built from single stem cells in the absence of a mesenchymal cellular niche. Subsequently, the addition of Wnt3a allowed for the generation of organoids from mouse colon crypts as well as purified Lgr5+ colonic stem cells. Furthermore, supplementation of niche factors Wnt3a, EGF, Noggin, and R-spondin-1, as well as the addition of nicotinamide, A83-01, a small-molecule inhibitor of transforming growth factor β (TGF-β) type I receptor kinase, also known as activin-like kinase 5 (ALK5), and SB202190, a p38 mitogen-activated protein kinase (MAPK) inhibitor, were required for the eventual establishment of long-term organoid cultures from primary adult human small intestinal or colonic epithelial tissue.

Modifications of the growth factors provided in the original intestinal organoid culture medium allowed establishment of epithelial organoid cultures from several other murine and human gastrointestinal organs over the past years, including gallbladder, liver, pancreas, and stomach. Eventually, using similar methods, organoid cultures were also generated from non-gastrointestinal epithelial tissues such as oesophagus, fallopian tube, mammary gland, lung, prostate, salivary gland and taste buds.

In 2011, establishment of intestinal organoid cultures from human ESCs and iPSCs was reported (Spence J R et al. (2011) Nature 470:105-109).

PSC-derived organoid cultures have also been generated for various other epithelial tissues such as inner ear, stomach, lung, liver and kidney.

Collectively, organoid cultures have been established from three different cellular sources (1) pure epithelial cells (single cells such as Lgr5+ stem cells or cell aggregates such as intestinal crypts), (2) minced tissue pieces containing both epithelial and mesenchymal cells, and (3) PSCs that are differentiated toward desired cell lineages.

### Crypts

The epithelial lining of the small and large bowel encompass luminal protrusions, villi (small bowel only), and invaginations, crypts. Each cell along the crypt-villus axis is polarised whereby cells on the top of the intestinal villi, or in the upper positions of colonic crypts, are the most differentiated and are continuously lost into the lumen. Continuous proliferation of stem cells residing in the basis of the crypts, and proliferation of progenitor cells residing in the middle of the crypts, ensures proper replacement of the shed cells.

Crypts can be isolated from the duodenum, small and large intestine, including jejunum, ileum, colon and rectum, and the pyloric region of the stomach by protocols that are known to the skilled person. For example, crypts can be isolated by incubation of isolated tissue with chelating agents that release cells from their calcium- and magnesium-dependent interactions with the basement membrane and stromal cell types. After washing the tissue, the epithelial cell layer is scraped from the submucosa with a glass slide and minced. This is followed by incubation in trypsin or, more preferred, EDTA and/or EGTA and separation of undigested tissue fragments and single cells from crypts using, for example, filtration and/or centrifugations steps. Other proteolytic enzymes, such as collagenase and/or dispase I, can be used instead of trypsin (as described in the methods herein). Similar methods are used to isolate fragments of the pancreas and other tissues. Methods to isolate stem cells are known and suitable methods for use with this disclosure can be selected by the skilled person depending on the stem cell type that is used. For example, isolation of epithelial stem cells may be performed using compounds that bind to Lgr5 and/or Lgr6, which are unique cell surface markers on epithelial stem cells. Examples of such compounds are anti-Lgr5 and anti-Lgr6 antibodies.

Cultured crypts undergo dramatic morphological changes after taking them into culture. The upper opening of freshly isolated crypts becomes sealed and this region gradually balloons out and becomes filled with apoptotic cells, much like apoptotic cells are pinched off at the villus tip or top end of a colonic crypt. The crypt region undergoes continuous budding events which create additional crypts, a process reminiscent of crypt fission. In one example, the organoids comprise crypt-like extensions which comprise all differentiated epithelial cell types, including proliferative cells, Paneth cells, enterocytes and goblet cells.

Expansion of the budding crypt structures creates organoids, comprising crypt-like structures surrounding a central lumen filled with apoptotic cell bodies.

A similar crypt organoid structure is formed when single epithelial stem cells are cultured. After about one week, structures are formed that strongly resemble the crypt organoid structures that are obtained with intact crypts.

In one example, the disclosure provides crypt organoids of the colon and rectum, or adenocarcinoma organoids of the colon or rectum generated or obtained by culturing human stem cells or tissue fragments according to a method of the disclosure. Such a population of organoids, for example, crypt colon or rectal organoids, generated or obtained by culturing human stem cells or tissue fragments according to a method of the disclosure, may each comprise more than 10, preferably more than 20, more preferably more than 40 organoids. The collection of organoids preferably comprises at least 10% viable cells, more preferred at least 20% viable cells, more preferred at least 50% viable cells, more preferred at least 60% viable cells, more preferred at least 70% viable cells, more preferred at least 80% viable cells, more preferred at least 90% viable cells. Viability of cells may be assessed using Hoechst staining or Propidium Iodide staining in FACS.

### Stem Cells

Stem cells are found in many organs of adult humans and mice. Although there may be great variation in the exact characteristics of adult stem cells in individual tissues, adult stem cells share the following characteristics: They retain an undifferentiated phenotype; their offspring can differentiate towards all lineages present in the pertinent tissue; they retain self-maintenance capabilities throughout life; and they are able to regenerate the pertinent tissue after injury. Stem cells reside in a specialized location, the stem cell niche, which supplies the appropriate cell-cell contacts and signals for maintenance of the stem cell population.

The types of stem cells amenable to culture according to the methods of the disclosure are embryonic stem cells, inducible stem cells (iPS) and adult stem cells. Unless, otherwise indicated, the stem cells herein are derived from adult tissue.

The stem cells of the disclosure cultured according to the disclosure may be human stem cells. In one example, the stem cells cultured according to the disclosure are epithelial stem cells.

Methods to isolate stem cells from epithelial tissue are known in the art. One method is based on the fact that stem cells express Lgr 5 and/or Lgr 6 on their surface, which belong to the large G protein-coupled receptor (GPCR) superfamily. The Lgr subfamily is unique in carrying a large leucine-rich ectodomain important for ligand binding. A suitable method therefore comprises preparing a cell suspension from the epithelial tissue, contacting the cell suspension with an Lgr5 and/or 6 binding compound, isolating the Lgr5 and/or 6 binding compound, and isolating the stem cells from the binding compound. It is preferred that a single cell suspension comprising the epithelial stem cells is mechanically generated from the isolated crypts.

In one example, the epithelial stem cells are isolated from large intestine fragments, gastric fragments or pancreatic fragments. For example, the epithelial stem cells are isolated from crypts that are isolated from the large intestine, including the colon or rectum, or the small intestine, including duodenum, jejunum and ileum.

In one example, the stem cells are human epithelial stem cells. Human epithelial stem cells include stem cells of human epithelial tissue origin. These include, but are not limited to pancreatic, small intestinal, large intestinal, corneal, olfactory, respiratory tissues, gastric tissues, liver and skin, for example, a tissue selected from the group consisting of pancreatic, small intestinal, large intestinal, corneal, olfactory, and respiratory tissues. Epithelial stem cells are able to form the distinct cell types of which the epithelium is composed. Some epithelia, such as skin or intestine, show rapid cell turnover, indicating that the residing stem cells must be continuously proliferating. Other epithelia, such as the liver or pancreas, show a very slow turnover under normal conditions.

The self-renewing epithelium of the small intestine is ordered into crypts and villi, while the epithelium of the colon and rectum is ordered into crypts (Gregoreff and Clevers, (2005) Genes Dev 19, 877-90). Each cell along the crypt-villus axis is polarized, whereby cells on the top of the villi of the small intestine, or in the upper positions of colonic crypts, are the most differentiated and are continuously lost into the lumen by apoptosis. Continuous proliferation of stem cells residing in the base of the crypts, and proliferation of progenitor cells residing in the middle of the crypts, ensures proper replacement of the shed cells. There is a resulting epithelial turnover time of approximately 5 days in the mouse. Self-renewing stem cells have long been known to reside near the crypt bottom and to produce the rapidly proliferating transit amplifying (TA) cells capable of differentiating towards all lineages. The estimated number of stem cells is between 4 and 6 per crypt (Bjerknes and Cheng, (1999) Gastroenterology 116, 7-14). Three differentiated cell types, enterocytes, goblet cells and enteroendocrine cells, form from TA cells and continue their migration in coherent bands along the crypt-villus axis. Each villus receives cells from multiple different crypts. The fourth major differentiated cell-type, the Paneth cell, resides at the crypt bottom.

Epithelial stem cells are able to form the distinct cell types of which the epithelium is composed. Some epithelia, such as skin or intestine, show rapid cell turnover, indicating that the residing stem cells must be continuously proliferating. Other epithelia, such as the liver or pancreas, show a very slow turnover under normal conditions.

In some examples of the disclosure, single Lgr5+ epithelial stem cells, for example from the colon, rectum, small intestine, or pancreas, may be used to form organoids, such as colonic, rectal, small intestinal or pancreatic organoids respectively.

In one example, the disclosure provides a population of cells or one or more organoids comprising stem cells or tissue fragments according to the disclosure, wherein the population has been cultured for at least 7 days, at least 14 days, at least 30 days, at least 3 months, at least 6 months, or at least 9 months.

A ‘population’ of cells is any number of cells greater than 1, but is preferably at least 1×10³ cells, at least 1×10⁴ cells, at least 1×10⁵ cells, at least 1×10⁶ cells, at least 1×10⁷ cells, at least 1×10⁸ cells, or at least 1×10⁹ cells.

### Culture of Stem Cells

Isolated stem cells are preferably cultured in a microenvironment that mimics at least in part a cellular niche in which the stem cells naturally reside. The cellular niche is mimicked by culturing the stem cells in the presence of biomaterials, such as matrices, scaffolds, and culture substrates that represent key regulatory signals controlling stem cell fate. The biomaterials comprise natural, semi-synthetic and synthetic biomaterials, and/or mixtures thereof. A scaffold provides a two-dimensional or three dimensional network.

As is known to a skilled person, the mechanical properties such as, for example, the elasticity of the scaffold influences proliferation, differentiation and migration of stem cells. It is furthermore preferred that the scaffold does not substantially induce an immunogenic response after transplantation into a subject. The scaffold is supplemented with natural, semi-synthetic or synthetic ligands, which provide the signals that are required for proliferation and/or differentiation, and/or migration of stem cells.

A cellular niche is in part determined by the stem cells and surrounding cells, and the extracellular matrix (ECM) that is produced by the cells in the niche. In an example of the disclosure, isolated crypts or epithelial stem cells are suspended in an ECM preparation. ECM is composed of a variety of polysaccharides, water, elastin, and glycoproteins, wherein the glycoproteins comprise collagen, entactin (nidogen), fibronectin, and laminin. ECM is secreted by connective tissue cells. Different types of ECM are known, comprising different compositions including different types of glycoproteins and/or different combination of glycoproteins. The ECM can be provided by culturing ECM-producing cells, such as for example fibroblast cells, in a receptacle, prior to the removal of these cells and the addition of isolated crypts or epithelial stem cells. Examples of extracellular matrix-producing cells are chondrocytes, producing mainly collagen and proteoglycans, fibroblast cells, producing mainly type IV collagen, laminin, interstitial procollagens, and fibronectin, and colonic myofibroblasts producing mainly collagens (type I, Ill, and V), chondroitin sulfate proteoglycan, hyaluronic acid, fibronectin, and tenascin-C.

Alternatively, the ECM is commercially provided. Examples of commercially available extracellular matrices are extracellular matrix proteins (Invitrogen), Matrigel® (BD Corning), Geltrex® (Thermo Fisher Scientific) or Cultrex® (Trevigen). The use of an ECM for culturing stem cells enhanced long-term survival of the stem cells and the continued presence of undifferentiated stem cells. In the absence of an ECM, stem cell cultures could not be cultured for longer periods and no continued presence of undifferentiated stem cells was observed. In addition, the presence of an ECM allowed culturing of three-dimensional tissue organoids, which could not be cultured in the absence of an ECM.

A preferred ECM for use in a method of the disclosure comprises at least two distinct glycoproteins, such as two different types of collagen or a collagen and laminin. The ECM can be a synthetic hydrogel extracellular matrix or a naturally occurring ECM. A preferred ECM is provided by Matrigel® (BD Corning), Geltrex® (Thermo Fisher Scientific) or Cultrex® (Trevigen), which comprises laminin, entactin, collagen IV and proteoglycans.

A cell culture medium that is used in a method of the disclosure comprises any cell culture medium. A preferred cell culture medium is a defined synthetic medium that is buffered at a pH of 7.4 (preferably between 7.2 and 7.6 or at least 7.2 and not higher than 7.6) with a carbonate-based buffer, while the cells are cultured in an atmosphere comprising between 5% and 10% CO₂, or at least 5% and not more than 10% CO₂, preferably 5% CO₂. An exemplary cell culture medium is selected from DMEM/F12 and RPMI 1640 supplemented with glutamine, insulin, penicillin/streptomycin and transferrin. In a further example, Advanced DMEM/F12 or Advanced RPMI is used, which is optimized for serum free culture and already includes insulin. In this case, the Advanced DMEM/F12 or Advanced RPMI medium is preferably supplemented with glutamine and penicillin/streptomycin. It is furthermore preferred that the cell culture medium is supplemented with a purified, natural, semi-synthetic and/or synthetic growth factor and does not comprise an undefined component such as fetal bovine serum or fetal calf serum. Supplements such as, for example, B27 (Invitrogen), N-Acetylcysteine (Sigma) and N2 (Invitrogen) stimulate proliferation of some cells and can further be added to the medium, if required.

Yet a further component that is added to the cell culture medium is one or more mitogenic growth factors selected from a family of growth factors comprising Epidermal Growth Factor (EGF; (Peprotech), Transforming Growth Factor-alpha (TGF-alpha; Peprotech), Basic Fibroblast Growth Factor (bFGF; Peprotech), Brain-Derived Neurotrophic Factor (BDNF; R&D Systems), and Keratinocyte Growth Factor (KGF; Peprotech). EGF is a potent mitogenic factor for a variety of cultured ectodermal and mesodermal cells and has a profound effect on the differentiation of specific cells in vivo and in vitro and of some fibroblasts in cell culture. The EGF precursor exists as a membrane-bound molecule which is proteolytically cleaved to generate the 53-amino acid peptide hormone that stimulates cells. A preferred mitogenic growth factor is EGF. EGF is preferably added to the culture medium at a concentration of between 0.5 and 100 ng/ml.

The FGF may be FGF10 or FGF7 or a combination thereof.

During culturing of stem cells, the mitogenic growth factor is preferably added to the culture medium every second day, while the culture medium is refreshed preferably every fourth day. Any member of the bFGF family may be used. Preferably, FGF7 and/or FGF10 is used. FGF7 is also known as KGF (Keratinocyte Growth Factor). In a further preferred embodiment, a combination of mitogenic growth factors such as, for example, EGF and KGF, or EGF and BDNF, is added to the basal culture medium. In a further example, a combination of mitogenic growth factors such as, for example, EGF and KGF, or EGF and FGF10, is added to the culture medium.

If required, additional components that may be added to the cell culture medium include a bone morphogenic protein (BMP) inhibitor e.g. Noggin, DAN, and DAN-like proteins including Cerebus and Gremlin; a Wnt agonist e.g. Wnt family member, R-spondin 1-4, Norrin and a GSK-inhibitor.

In other examples, the culture medium may comprise a Notch agonist. Notch signalling has been shown to play an important role in cell-fate determination, as well as in cell survival and proliferation.

### Hydrogels

A hydrogel is a three-dimensional network of hydrophilic polymers that can swell in water and retain water while maintaining the structure due to chemical or physical cross-linking of individual polymer chains. By definition, water must constitute at least 10% of the total weight (volume) for a material to be a hydrogel. Hydrogels also possess a degree of flexibility very similar to natural tissue due to their significant water content.

In some examples, the hydrogel is a natural material. For example, the hydrogel may be agarose, gelatin, chitosan, alginate, fibrin, methycellulose, hyaluronan, an elastin-like polypeptide or other naturally derived polymer.

Synthetic hydrogels suitable for use in the methods of the present disclosure include homopolymers, co-polymers, and interpenetrating network hydrogels. Homopolymeric hydrogels are referred to as a polymer network derived from a single species of monomer. Homopolymers may have cross-linked skeletal structure depending on the nature of the monomer and polymerization technique. Co-polymeric hydrogels are comprised of two or more different monomer species with at least one hydrophilic component, arranged in a random, block or alternating configuration along the chain of the polymer network. Multipolymer Interpenetrating polymeric hydrogel (IPN), is made of two independent cross-linked synthetic and/or natural polymer component, contained in a network form. In semi-IPN hydrogel, one component is a cross-linked polymer and other component is a non-cross-linked polymer.

Persons skilled the art will be familiar with the process for making synthetic hydrogels. Non-limiting examples of suitable monomeric units that can be used in the formation of homo and co-polymers include N-vinyl-2-pyrrolidone, N-isopropyl acrylamide, vinyl acetate, acrylic acid, methacrylic acid (MAA), poly(2-hydroxyethyl methacrylate) (PHEMA), 2-Hydroxyethyl methacrylate (HEMA) and polyethylene glycol (PEG).

### Utility of Organoids

In a further example, the disclosure provides the use of the normal and tumour organoids, (e.g. colon, pancreas, breast, lung etc.) as described herein in a drug discovery screen, drug testing assays and toxicity assays.

The organoids as described herein are also useful for functional assays, co-culture assays, transplantation assays and to examine the production of biological products.

The organoids as described herein are also useful for infection assays and pathogen expansion assays.

The organoids as described herein are also useful for genetic testing assays, gene editing assays and regenerative medicine.

For high-throughput purposes, the organoids are cultured in multiwell plates such as, for example, 96 well plates 384 well plates or 1536 well plates. Libraries of molecules are used to identify a molecule that affects the organoids. Preferred libraries comprise antibody fragment libraries, peptide phage display libraries, peptide libraries (e.g. LOPAP™, Sigma Aldrich), lipid libraries (BioMol), synthetic compound libraries (e.g. LOPAC™, Sigma Aldrich) or natural compound libraries (Specs, TimTec). Furthermore, genetic libraries can be used that induce or repress the expression of one of more genes in tumour or normal organoids.

These genetic libraries comprise CRISPR libraries, cDNA libraries, antisense libraries, and siRNA or other non-coding RNA libraries. The cells are preferably exposed to multiple concentrations of a test agent for certain period of time. At the end of the exposure period, the cultures are evaluated.

The term “affecting” is used to cover any change in a cell, including, but not limited to, a reduction in, or loss of, proliferation, a morphological change, and cell death. Organoids derived from normal tissues can also be used to identify drugs that specifically target tumour organoids, but not the normal organoids.

The normal tissue derived organoids can further replace the use of cell lines such as Caco-2 cells in toxicity assays of potential novel drugs or of known or novel food supplements.

Furthermore, the normal tissue derived organoids can be used for culturing of a pathogen such as a norovirus which presently lacks a suitable tissue culture or animal model.

Cultures comprising normal tissue derived organoids are useful in regenerative medicine, for example in post-radiation and/or post-surgery repair of the intestinal epithelium, in the repair of the intestinal epithelium in patients suffering from inflammatory bowel disease such as Crohn's disease and ulcerative colitis, and in the repair of the intestinal epithelium in patients suffering from short bowel syndrome. Further use is present in the repair of the intestinal epithelium in patients with hereditary diseases of the small intestine/colon.

It will be clear to a skilled person that gene therapy can additionally be used in a method directed at repairing damaged or diseased tissue. Use can, for example, be made of an adenoviral or retroviral gene delivery vehicle to deliver genetic information, like DNA and/or RNA to stem cells. A skilled person can replace or repair particular genes targeted in gene therapy. For example, a normal gene may be inserted into a nonspecific location within the genome to replace a non-functional gene. In another example, an abnormal gene sequence can be replaced for a normal gene sequence through homologous recombination. Alternatively, selective reverse mutation can return a gene to its normal function. A further example is altering the regulation (the degree to which a gene is turned on or off) of a particular gene. Preferably, the stem cells are ex vivo treated by a gene therapy approach and are subsequently transferred to the mammal, preferably a human being in need of treatment.

In addition, the disclosure provides for the use of tumour organoids for a targeted drug discovery screen to identify a drug that specifically affects tumour cells compared to expanded normal cells that are cultured with the same drug. For high-throughput purposes, the tumour organoids cultured in multiwell plates such as, for example, 96 well plates, 384 well plates or 1536 well plates. Libraries of molecules are used to identify a molecule that affects the tumour organoids. Preferred libraries comprise antibody fragment libraries, peptide phage display libraries, peptide libraries (e.g. LOPAP™, Sigma Aldrich), lipid libraries (BioMol), synthetic compound libraries (e.g. LOP AC™, Sigma Aldrich) or natural compound libraries (Specs, TimTec). Furthermore, genetic libraries can be used that induce or repress the expression of one of more genes in the progeny of the tumour cells. These genetic libraries comprise CRISPR libraries, cDNA libraries, antisense libraries, and siRNA or other non-coding RNA libraries. A compound that affects tumour cells may subsequently, or in parallel, be tested for affecting expanded organoids from matched normal cells. The term “affecting” is used to cover any change in a cell, including a reduction in, or loss of, proliferation, a morphological change, and cell death. the progeny can also be used to identify drugs that specifically target epithelial tumour cells, including reversion of the tumour cells.

It will be clear that the tumour or normal organoids can also be used in a high throughput approach for the determination of in vitro metabolic stability and metabolic profiles of drug candidates.

The disclosure further provides for the use of the tumour organoids according to the disclosure and of normal tissue derived organoids of the disclosure, in toxicity assays. the tumour and normal tissue derived organoids are easy to culture and more closely resemble primary epithelial cells than, for example, epithelial cell lines such as Caco-2 (ATCC HTB-37), 1-407 (ATCC CCL6), and XBF (ATCC CRL 8808) which are currently used in toxicity assays. It is anticipated that toxicity results obtained with primary tumour cultures or with crypt-organoids more closely resemble results obtained in patients. A cell-based toxicity test is used for determining organ specific cytotoxicity. Compounds that are tested in the test comprise cancer chemopreventative agents, environmental chemicals, food supplements, and potential toxicants. The cells are exposed to multiple concentrations of a test agent for certain period of time. The concentration ranges for test agents in the assay are determined in a preliminary assay using an exposure of five days and log dilutions from the highest soluble concentration. At the end of the exposure period, the cultures are evaluated for inhibition of growth. Data may be analysed to determine for example the concentration that inhibited end point by 50 percent (ED₅₀).

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### Materials and Methods

### Matrigel

Matrigel® matrix is a reconstituted basement membrane preparation (gelatinous protein mixture) extracted from the Engelbreth-Holm-Swarm (EHS) mouse sarcoma, a tumour rich in extracellular matrix proteins. This material, once isolated, is approximately 60% laminin, 30% collagen IV, 8% entactin and 2% proteoglycans. Entactin is a bridging molecule that interacts with laminin and collagen IV and contributes to the structural organisation of these extracellular matrix molecules. Matrigel also contains heparin sulphate proteoglycan (perlecan), transforming growth factor beta (TGF-β), epidermal growth factor (EGF), insulin-like growth factor (IGF-1), basic fibroblast growth factor (bFGF), nerve growth factor (NGF), platelet derived growth factor (PDGF), tissue plasminogen activator and other growth factors which occur naturally in the EHS tumour. There is also residual matrix metalloproteinases derived from the tumour cells.

Matrigel is available commercially from a number of different sources. Matrigel used for the present studies was purchased from BD Corning Life Sciences (catalog number 354248).

Typical concentrations of the growth factor components of Matrigel are described in Table 1 below.

TABLE 1
Growth factor concentrations in Matrigel

| | Range of GF | |
|---|---|---|
| Growth Factor | concentration | |
| Epidermal Growth factor (EGF) | 0.5-1.3 | ng/ml |
| Basic Fibroblast Growth factor (bFGF) | <0.1-0.2 | pg/ml |
| Nerve Growth factor (NGF) | <0.2 | ng/ml |
| Platelet derived Growth factor (PDGF) | 5-48 | pg/ml |
| Insulin-like Growth factor-1 (IGF-1) | 11-24 | ng/ml |
| Transforming Growth factor-beta (TGF-β) | 1.7-4.7 | ng/ml |

### Cancer Organoid Medium

The Matrigel (typically provided at a protein concentration of between 8 to 11 mg/ml) is diluted to a concentration range of between 3-5% (0.24 mg/ml to 0.55 mg/ml) in either Cancer Organoid medium or Normal Organoid medium.

The reduced cancer Organoid Medium comprises DMEM/F12 (Gibco)+20 ng/mL basic fibroblast growth factor (Gibco)+50 ng/mL epidermal growth factor (Peprotech), 0.01M nicotinamide (Sigma), 1 mM N-acetyl-L-cysteine (Sigma), 1×B27 supplement (Gibco), 1×N2 supplement (Gibco), 1×HEPES buffer (Gibco), 1× penicillin/streptomycin, primocin or normocin.

The Normal Organoid Medium comprises Intesticult™ (Stem Cell Technologies catalog number 06005), primocin or normocin.

For the growth of breast cancer stem cells, the Cancer Organoid Medium was used as described above.

### Patient Samples

Tumor and adjacent normal tissue samples were obtained from patients with colorectal adenocarcinoma with appropriate ethical approvals. This study was conducted in accordance with the Declaration of Helsinki, the NHMRC Statement on Ethical Conduct in Human Research and institutional human research ethics approval (HREC 2016.249). All patients gave informed consent. For patients undergoing resection of primary tumors, organoids were generated from tumor tissue and adjacent normal tissue from the resection margin. For patients undergoing resection of metastatic disease, tumor organoids were generated from the site of metastasis. Specimens with a volume of greater than 5 mm³ were collected at surgery and placed into collection medium containing DMEM/F12 (Life Technologies, 11320082), penicillin-streptomycin (Life Technologies, 15140122), gentamycin (Merck, G1397), kanamycin (Merck, K0254), amphotericin B (Merck, A2942) and nystatin (Merck, N1638), and stored for up to 72 h at 4° C. before processing.

### Preparation of Organoids

Normal or tumour tissue specimens are collected at surgery, washed in ice-cold phosphate buffered saline (PBS) and treated with 0.4-0.1% (preferably 0.1%) hyperchlorite in PBS for 10-20 min at room temperature. Samples are washed in ice-cold PBS to remove residual sodium hyperchlorite solution.

Normal organoids were prepared by incubation of tissue in 3 mM ethylenediaminetetraacetic acid (EDTA) chelation buffer (PBS/3 mM EDTA/100 μM dithiothreitol) for 30-60 min at room temperature. Tissue was removed and placed into PBS and shaken to release colorectal epithelium. Crypts were harvested by centrifugation of supernatant at 800 rpm for 1 min. Isolated colonic crypts were resuspended in 2 ml digestion buffer (0.1 mg/mL of dispase in DMEM/F12 medium) and incubated at 37° C. for 5-10 min. Crypts were broken up by manual pipetting and centrifuged at 800-1,000 rpm for 1 min. The cell pellet was resuspended in DMEM/F12 medium and centrifuged at 800-1,000 rpm for 1 min. Crypt fragments were resuspended in Normal Organoid Medium comprising 3-5% of Matrigel matrix and supplemented with 250 U/ml penicillin/streptomycin and 10 μM0111 Y27632 and incubated at 37° C. with 5-10% CO₂ for two days. Organoids were subsequently propagated in Normal Organoid Medium comprising 3-5% of Matrigel matrix, with regular agitation to prevent organoid precipitation. Media changes (50:50) were conducted every 2-7 days.

Examples 1 to 4: colon cancer tumour organoids were prepared by incubation of tissue in 5 ml Digestion buffer (0.1 mg/ml Dispase, 200 U/ml collagenase IV (Sigma)) in DMEM/F12 media and processed using a cell dissociator (gentle MACS Dissociator) with 3 cycles of 37 sec mincing followed by incubation at 37° C. with 5-10% CO₂ for 10 min. Digested tissue was filtered through a 70 μm strainer to remove large debris and centrifuged at 1000-1600 rpm for 3 min. The cell pellet was resuspended in DMEM/F12 media and centrifuged at 1000-1600 rpm for 3 min. Tumour fragments were resuspended in Cancer Organoid Medium comprising 3-5% of Matrigel matrix and supplemented with 250 U/ml penicillin/streptomycin, 500 μg/ml nystatin and 10 μM Y27632 and incubated at 37° C. with 5-10% CO₂ for two days. Organoids are subsequently propagated in Cancer Organoid Medium comprising 3-5% of Matrigel matrix, with regular agitation to prevent organoid precipitation. Media changes (50:50) were conducted every 2-7 days.

Breast cancer organoids were prepared as described above.

Examples 5 to 11: for normal colorectal tissue, specimens were incubated in 3 mM EDTA chelation buffer (Sigma-Aldrich, E5134) containing 100 μM dithiothreitol (Merck, 10197777001) for 30-60 min at room temperature, transferred into PBS and shaken vigorously to release crypts. Crypts were collected by centrifugation and digested with 0.1 mg/ml dispase (Life Technologies, 17105-041) in DMEM/F12 to produce fragments of crypts. Samples were washed with DMEM/F12. Crypt fragments were resuspended in IntestiCult Organoid Growth Medium (Human) (Stemcell Technologies, 06010) containing 10 μM Y27632 (Stemcell Technologies, 72308), penicillin-streptomycin (Life Technologies, 15140122) and primocin (Invivogen, ant-pm-1).

Tumor samples were digested with 0.1 mg/ml dispase II (Merck, D4693-1G) and 200 U/mL collagenase IV (Life Technologies, 17104019) in DMEM/F12 using a gentleMACS™ Dissociator (Miltenyi Biotec, 130-093-235). Samples were washed with DMEM/F12 and fragments were collected by centrifugation. The tissue fragments were filtered through a 70 μm cell strainer (Bio-Strategy, BDAA352350), washed with DMEM/F12 and resuspended in “complete” medium containing DMEM/F12, HEPES (Sigma-Aldrich, H3375-1KG), B27 supplement (LifeTechnologies, 17504001), N2 supplement (Life Technologies, 17502001), nicotinamide (Sigma-Aldrich, 72340-1KG), N-acetyl-L-cysteine (Sigma-Aldrich, A7250-1KG), Wnt3A-conditioned medium 50% (vol/vol) (harvested from Wnt3A-expressing L cell line; ATCC, CRL-2647), recombinant human R-spondin-2 (PeproTech, 120-43), recombinant human EGF (Life Technologies, PHG0313), recombinant human noggin (PeproTech, 120-10), A83-01 (Tocris Bioscience, 29-391-0), SB202190 (Sigma-Aldrich, S7067), penicillin-streptomycin and primocin, or “reduced” medium containing only DMEM/F12, HEPES, B-27 supplement, N2 supplement, nicotinamide, N-acetyl-L-cysteine, recombinant human basic FGF (Life Technologies, PHG0263), and recombinant human EGF; 10 μM Y27632 was added at initial seeding.

For organoid establishment in donut culture 24, 15 μl of Matrigel matrix (Bio-strategy, BDAA354234), chilled at 4° C., was used to generate a ring around the circumferences of a well in a 96-well plate. The Matrigel ring was solidified at 37° C. for 30 min and then overlaid with culture medium containing tissue fragments.

For dome culture, fragments were suspended in Matrigel matrix and plated as 30 μl domes in 6-well plates with a density of 8 domes per well. Matrigel domes were solidified by incubation at 37° C. for 30 min, followed by addition of culture medium. Organoids were established in a CO₂ incubator (Thermo Fisher Scientific) at 37° C. with 5% CO₂.

For organoid establishment in LVM suspension culture, fragments were suspended in media containing 5% Matrigel or other 5% matrix preparations, including BME-1 (Basement Membrane Extract, Type 1, PathClear, Cultrex, 3432-010-01), BME-2 (Basement Membrane Extract, Type 2, PathClear, Cultrex, 3532-010-02) and collagen type I-A (Cellmatrix Collagen Type I-A, Nitta Gelatin Inc., 631-00651), and plated in 6-well suspension plates (Interpath, 657185) or Corning™ Mini Bioreactor Centrifuge Tubes (Corning, 431720). Culture medium was changed every 2-3 days with agitation of organoids by pipetting.

### Organoid Propagation

Organoid cultures were passaged when organoids reached a diameter of 100 to 300 μm. For donut and dome cultures, organoids were scraped off plates with pipette tips and collected; for LVM suspension cultures, organoids were harvested via pipette aspiration. Organoids were washed in PBS and digested with TrypLE Express enzyme (Thermo Fisher Scientific, 12604021) for 10-20 min at 37° C. Digestion was terminated with 1% BSA in DMEM/F12 and organoids were pelleted by centrifugation. Pellets were resuspended in culture medium and mechanically broken into fragments with T200 pipette tips or single cells with 26 G needles. Cells or fragments were subsequently replated via dome or LVM suspension culture methods, as described above. Organoid cultures were tested for absence of mycoplasma using the LookOut Mycoplasma PCR Detection Kit (Sigma-Aldrich, MP0035-1KT). For biobanking, small (50 to 100 μm) organoids were harvested, washed and suspended with DMEM/F12, combined with CryoStor 10 medium (STEMCELL Technologies, 7930), frozen in CoolCell LX (Biocision, BCS-405) at −80° C. and transferred into liquid nitrogen for long-term storage.

### Bright-Field Microscopy

Bright-field microscopy to document organoid growth overtime was performed on an Eclipse TS100 (Nikon) microscopy systems with a 2× or 10× objective and a TrueChrome camera and TCapture software or an Eclipse Ti-U (Nikon) microscopy systems with 4× or 10× objective and a DS-Ri2 camera and NIS-Elements BR software.

### Histology

Organoids were harvested, mixed with HistoGel (Themo Fisher Scientific, 22-110-678), dropped onto 6-well plates to generate domes and solidified at 4° C. for 15 min. Prepared domes were fixed in 10% Formalin for 30 min and stored in PBS at 4° C. prior to embedding in paraffin. Normal and tumor tissue samples from patients with colorectal cancer were embedded in Tissue-Tek O.C.T Compound (Emgrid, 4583) and rapidly frozen using an isopropyl bath surrounded by dry ice. Specimen sections were prepared for hematoxylin and eosin (H&E) staining. Stained slides were scanned on a 3D Histech Panoramic Scan II histology scanner (3DHISTECH Ltd.) and examined in CaseViewer Software (3DHISTECH Ltd.).

### Immunofluorescence Microscopy

Organoids were harvested, washed with PBS, fixed with 10% formalin for 60 min at 4° C., permeabilized in 0.5% Triton X-100 in PBS for 30 min and blocked in 1% BSA in PBS for 1 h at room temperature or overnight at 4° C. Organoids were thoroughly washed with washing buffer (0.2% TritonX-100 and 0.05% Tween20 in PBS) and incubated with primary antibodies: anti-Ki67 antibody (Abcam, ab92742, 1:50), or anti-E-Cadherin antibody (Abcam, ab1416, 1:50) in 0.2% BSA in PBS at 4° C. overnight. Organoids were washed with washing buffer and incubated with secondary antibodies (1:400): Alexa Fluor 488 Goat anti-rabbit IgG (Invitrogen, A11008) or Alexa Fluor 488 Goat anti-mouse IgG (Invitrogen, A11001) in 0.2% BSA in PBS at 4° C. overnight. Organoids were washed with washing buffer and F-actin was stained with Phalloidin (Alexa Fluor™ 546 Phalloidin, Invitrogen, A22283.1:80) in PBS for 30 min at room temperature followed by a wash in PBS. Prior to imaging, nuclei were stained with DAPI (Sigma. MBD0015, 1:1000) for 10 min at room temperature and residual DAPI was washed off with PBS. Organoids in PBS were transferred into a p-Slide 8 Well slide (Ibidi, 80826) and images were captured using a Leica SP8 Confocal microscope with a 40× objective and Leica LAS×LS software.

### Whole Genome Sequencing

A total of 26 cancer organoids were sequenced on a DIPSEQ platform (BGI). Pre-processing, including removal of low-quality reads and adaptor sequences, was carried out using SOAPnuke (v2.0.7) 25. High quality reads were mapped and processed for downstream analysis using Sentieon Genomics software (version: sentieon-genomics-201911) 26 which includes the following optimized steps: 1) aligned hg38 with BWA MEM 27 with alt-aware mapping model; 2) sorted alignment reads by Samtools 28; 3) marked duplicate reads by Picard; 4) indel realignment and base quality score recalibration for alignment reads by GATK 29. 5) alignment QC by Picard.

For paired normal and cancer organoids, somatic SNVs were identified using five variant callers, including Mutect2 (GATK v4.0.10.1), Strelka2 (v2.9.9), MuSE (v1.0), SomaticSniper (v1.0.5.0), and Lancet (v1.0.7). Somatic INDELs were identified using four tools, including MuTect2 (GATK v4.0.10.1), Strelka2 (v2.9.9), Lancet (v1.0.7), and Svaba (v0.2.1). High-confidence somatic SNVs and INDELs were retrained and annotated when identified by at least two tools. SNVs and INDELs were annotated by ANNOVAR (v20180416). Somatic CNVs were identified by cnv_facets (v0.16.0) (Shen, R. & Seshan, V. E. FACETS: allele-specific copy number and clonal heterogeneity analysis tool for high-throughput DNA sequencing. Nucleic Acids Res 44, e131 (2016). For single cancer organoid samples, putative somatic SNVs and INDELs were identified and filtered by Mutect2 and FilterMutectCalls (GATK v4.0.10.1) in tumor-only mode. Putative somatic mutations were identified by filtering out SNVs and INDELs found with a 0.0001 or higher frequency in the Genome Aggregation Database (Karczewski, K et al (2020) Nature. 581, 434-443) and 0.0005 or higher frequency in the 1000 Genomes Project (Genomes Project, C et al (2015) Nature. 526, 68-74) as well as polymorphisms identified in five reference normal samples sequenced on the same platform. Somatic SNVs and INDELs were annotated by Personal Cancer Genome Reporter (PCGR) (v0.8.1) (Nakken, S. et al. (2018) Bioinformatics 34, 1778-1780).

### Semiautomated High-Throughput Drug Assay

Organoids were digested with TrypLE Express enzyme at 37° C. for 15-20 min. Digestion was terminated in 1% BSA in DMEM/F12 and fragments were collected by centrifugation. Pellets were resuspended in culture medium, dissociated with a 26 G needle and filtered through a 40 μm cell strainer (Bio-Strategy, BDAA352340) to produce a single cell suspension. Live cells were counted using a hemocytometer and trypan blue staining. Single cells (3000 cells in 60 μl/well) were suspended in culture medium with 3% Matrigel, penicillin-streptomycin, normocin and 10 μM Y27632 and seeded in 384-well optical plates (Thermo Fisher Scientific, NUN242764) using a MANTIS® liquid handling robot (Formulatrix), and established for 3 days prior to drug addition. For plate uniformity studies, 1 μM bortezomib (Selleck, S1013) was used as the positive control, and 0.5% DMSO (Sigma, D2650) was used as a negative vehicle control. For the drug-dose response curves, regorafenib (Selleck, S1178) was used in a 9 point 4-fold titration series starting at 50 μM. All compound plates were prepared using a JANUS liquid handling robot (PerkinElmer) and stamped into 384-well plates using an integrated PinTool addition accessory (PerkinElmer).

For drug sensitivity testing of standard-of-care chemotherapy agents for colorectal cancer, drugs were assayed in duplicate in a 9 point, 4-fold dilution series with starting concentrations of 50 μM for 5-fluorouracil, 75 μM for oxaliplatin, 0.5 μM for SN-38, 50 μM for regorafenib and 50 μM for TAS-102. 0.5% DMSO and 1 μM bortezomib served as negative and positive controls, respectively.

PDTOs were imaged every 24 h for 7 days by bright-field microscopy as z-stacks on an automated Eclipse Ti2 Inverted Microscope System with an integrated tissue culture incubator (Nikon). 384-well images were captured using a 4× dry objective and Nikon Eclipse Ti2 software with a 25 μm Z-stack over a range of 500 μm. On day 7, 20μl CellTiter-Glo 3D reagent (Promega, G9683) was added using the MANTIS® liquid handling robot, plates were shaken at 1000 rpm for 30 mins at room temperature and luminescence measured using an EnVision plate reader (PerkinElmer).

### Computational Image Analysis

For image analysis, flattening of Z-stack bright-field images, removal of background debris and selection of organoids were performed using ImageJ software (NIH Image). The projection of z-stack images onto a single in-focus image was carried out using the Extended Depth of Field (EDF) plugin in “easy mode”, with the parameter quality=′1′ and topology=1′. Processed images were exported as high-quality tiff files. Background debris removal was performed by with a series of steps including Subtract Background, Enhance Local Contrast (CLAHE), set AutoThreshold, set Black Background, and Convert to Mask. Organoid selection and feature extraction were achieved using sequential Analyze Particles, Invert LUT, Fill Holes, Watershed, Set Measurements and Analyze Particles steps. Features of selected organoids were exported as text files for determination of mean organoid size.

### Dose Response Quantification

Growth rate-adjusted organoid viability (GR) values were calculated for imaging data based on day 0 negative control, day 7 negative/positive control, and day 7 drug-treated wells described by Hafner et al using the following equation (Hafner, M., Niepel, M., Chung, M. & Sorger, P. K. (2016) Nat Methods 13, 521-527).

$$GR ( c ) = \cdot {2}^{( l o {g}_{2} ( x ( o )} {\text{?}}^{{x}_{0} )} {\text{?}}^{l o {g}_{2} ( {x}_{Ctrl}} {\text{?}}^{{x}_{0} ) )} - 1. ¶$$

$$\text{?} \text{indicates text missing or illegible when filed}$$

Where x(c) is the mean organoid size for treated wells at the end point, xCtrl is the mean organoid area of DMSO controls at the end point and x0 is the mean organoid area of control wells at time 0. Mean size of organoid debris remaining in positive (killing) control wells was subtracted from these values. Growth rate adjusted response curves were fitted using a 4-parameter log-logistic function in the “DRC” package. GR50 (concentration of drug that reduces cell proliferation rate by one-half), GRmax (GR value at maximum dose) and GRAOC (area over curve) were calculated as described by Hafner et al supra.

For comparison of uniformity of regorafenib dose response curves between imaging and CellTitreGlo 3D data, response curves were fitted using a 4-parameter log-logistic function to percent viability data.

### Evaluation of Assay Quality

Organoid assays in 384-well plates were examined for quality using four metrics:

(i) Coefficient of variation: CV=s.d./mean×100%
(ii) Z′ factor: Z′=1−3×(s.d.(Cpos)+s.d.(Cneg))/abs(mean(Cpos)−mean(Cneg))
(iii) Robust Z′ factor: RZ′=1−3×(m.a.d.(Cpos)+m.a.d.(Cneg))/abs(median(Cpos)−median(Cneg))
(iv) Signal window: SW=(abs(mean(Cpos)−mean(Cneg))−3×(s.d.(Cpos)+s.d.(Cneg)))/s.d.(Cpos) Assays meeting criteria of CV<20%, standard Z′ or robust Z′>0.4 and SW>2 were defined as acceptable (Iversen, P. W., Eastwood, B. J., Sittampalam, G. S. & Cox, K. L. (2006) J Biomol Screen 11, 247-252).

### Example 1 Organoid Growth Efficacy in Low-Viscosity Matrigel

The 3-D matrix produced by solid-state Matrigel is considered a key requirement for the formation of normal and tumour organoids. The matrix that is traditionally used for the growth of normal and tumour organoids was replaced with low-viscosity 3-5% Matrigel matrix which, unlike traditional Matrigel does not solidify at 37° C. A total of 3.5 mL of both Normal or Cancer Organoid Media comprising low-viscosity 3-5% Matrigel matrix containing 100,000 cells was cultured in a well of a 6 well-plate. For the dome culture, 100,000 cells were suspended in 240 uL of Matrigel and seeded as domes (30 uL) in a well of 6 well-plate and 3.5 mL of medium was added.

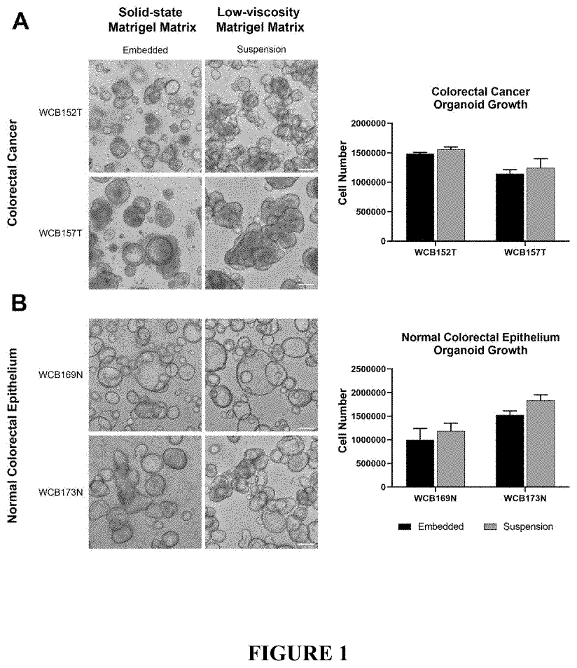

Organoids were grown over 14 days, with regular agitation (low-viscosity suspension) and media changes (50:50) every 2-5 days. Organoids grown in the low-viscosity 3-5% Matrigel matrix produced comparable numbers of cells to the traditional solid-state Matrigel matrix (FIG. 1).

### Example 2 Drug Sensitivity Testing of Colorectal Cancer Organoids

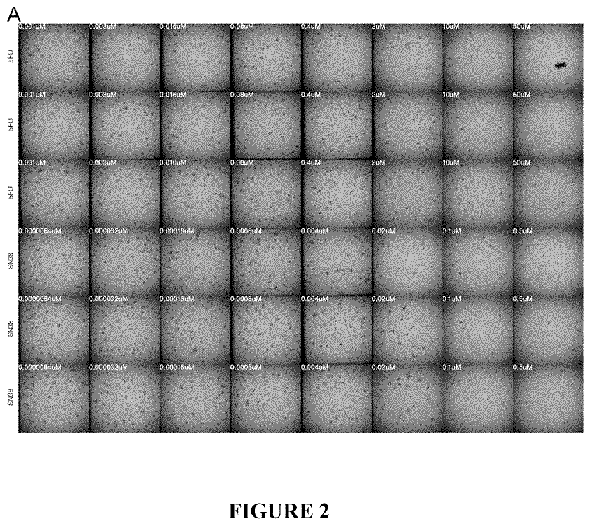

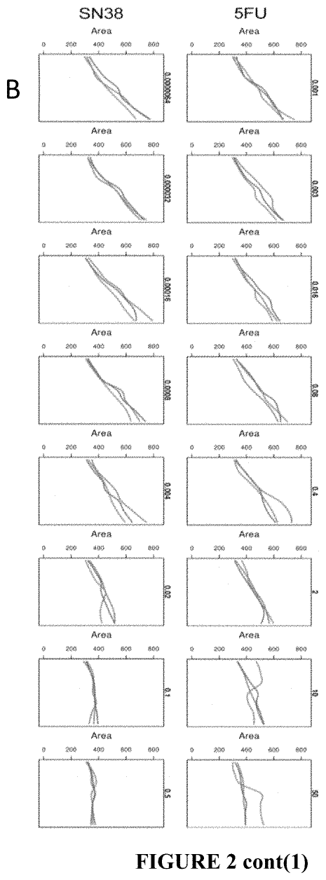

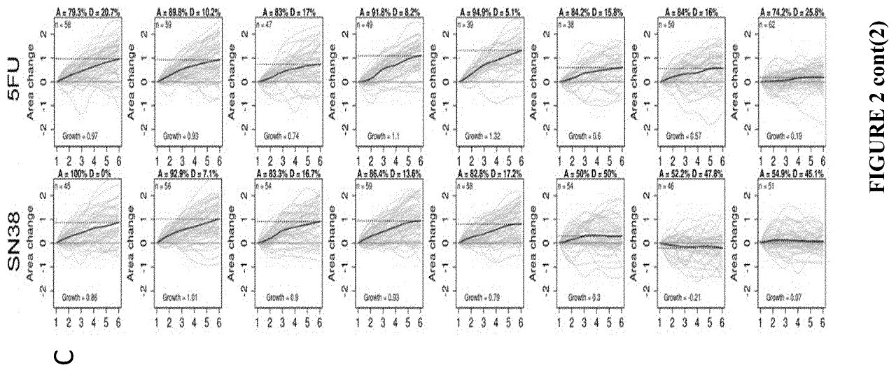

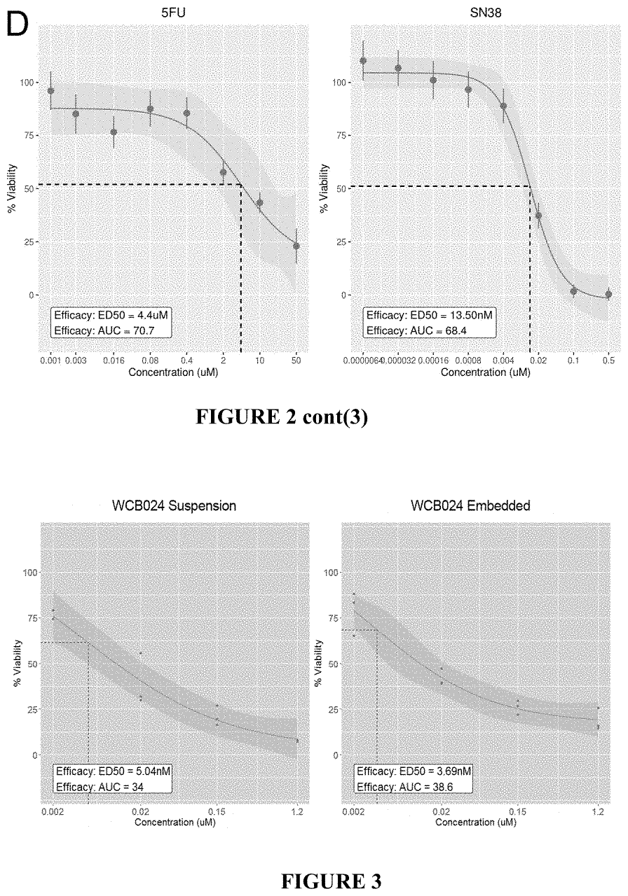

Organoids were established from single cells in Cancer Organoid Medium comprising 3-5% of Matrigel matrix. Single cells were dispensed into 384-well optical plates at 3,000 cells per well using a MANTIS® Microfluidic Liquid Handler. Organoids were allowed to establish for three days, followed by the addition of 5-fluoruracil (5-FU) or SN38 (active metabolite of irinotecan) as a 5-fold, 8-point dose titration in triplicate (DMSO was used as vehicle control). Organoid growth was recorded by daily bright-field microscopy imaging for six days. Representative images of organoids at day 6 post drug addition for triplicate wells are shown in FIG. 2A. Growth of organoids for triplicate wells over six days was quantified using image analysis in FIG. 2B. Individual organoid growth is tracked over time and quantified using image analysis in FIG. 2C. The low-viscosity of the Matrigel matrix thus facilitates liquid-handling applications such as drug sensitivity testing removing the need for a traditional solid Matrigel matrix application of which has cooling requirements. Even at this low viscosity, the Matrigel matrix remained sufficient to “fix” organoids in 3-D space, enabling tracking of individual organoids by microscopy imaging over prolonged time periods. FIG. 2D shows the dose-response curves and ED50 determination for the colorectal cancer organoids at 6 days post addition of 5-FU and SN38 based on the acquired images.

Growth in low-viscosity 3% Matrigel matrix produced ED50 values from drug dose response curves that were comparable to those derived from drug assays conducted with organoids embedded in solid-state Matrigel matrix (FIG. 3).

This approach has been found to be ideal for automated organoid passaging and drug testing applications.

### Example 3 Large-Scale Expansion Cultures for Colorectal Cancer Organoids

A bottleneck of current organoid propagation techniques using solid matrices is challenging organoid expansion, due to the technical requirements for preparation of such matrices and the need to extract embedded organoids for propagation.

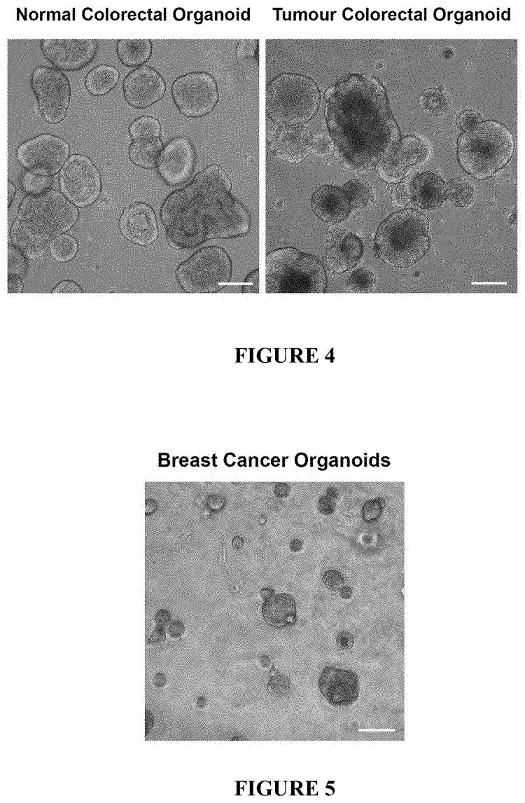

A total of 7. 5 ml of cell suspension containing 200,000 cells was cultured in 50 ml Mini Bioreactor Tubes (Corning) for 14 days with regular gentle agitation and media change (50:50) every 2-5 days, resulting in effective organoid growth. This method therefore enables substantial organoid expansion within a low passage number, a requirement for maintenance of organoid genomic fidelity and necessary for implementation of high-throughput screening or production applications (FIG. 4).

### Example 4 Examples of Organoid Cultures from Human Breast Cancer

Breast cancer organoids were suspended as single cells (100,000 cells in 3.5 ml) in Cancer Organoid Media comprising 3-5% of Matrigel matrix. Organoids were grown over 14 days, with regular agitation and media changes (50:50) every 2-5 days. Organoids grew efficiently in the low-viscosity 3-5% Matrigel matrix (FIG. 5). The use of low-viscosity Matrigel matrix was therefore readily transferable across different malignancies.

### Example 5 Selection and Optimisation of Organoid-Culture Media for Colorectal Normal and Cancer Tissues

The culture conditions for normal epithelial organoids from the human large intestine are well-defined (Sato, T. et al. (2011) Gastroenterology 141, 1762-1772. A typical formulation of growth medium (termed “complete” medium) may include DMEM/F12, HEPES, B27 supplement, N2 supplement, nicotinamide, N-acetyl-L-cysteine, Wnt3A, R-spondin-2, EGF, noggin, A83-01, SB202190, penicillin-streptomycin and primocin (Sato T et al., (2011) supra; Sato, T. et al. (2009). Nature 459, 262-265). Wnt3A can be provided as a conditioned medium sourced from a Wnt3a-producing cell line, but this contains undefined components from accompanying serum. Purified Wnt3A protein rapidly loses activity in culture media due to its hydrophobic nature, however this can be overcome with the use of phospholipids and cholesterol as carriers. A corresponding quality controlled intestinal organoid culture medium is commercially available (IntestiCult Organoid Growth Medium (Human), Stemcell Technologies).

The niche factor requirements for intestinal cancer organoids are more adaptable, with Wnt3A and R-spondin commonly omitted to enable selective outgrowth of WNT pathway mutated tumor cells (Kuhnert, F. et al. (2004) Proc Natl Acad Sci USA 101, 266-271). Considering that media supplementation with growth factors and inhibitors constitutes a major cost for organoid applications, we evaluated a reduced medium for cancer organoid culture, omitting Wnt3A, R-spondin, BMP, TGF-beta and p38 inhibitors from the complete medium formulation and supplementing with bFGF.

To compare utility of these media for cancer organoid culture, success rates were examined for a consecutive series of 64 colorectal cancer specimens, 28 of which were cultured in complete medium and 36 of which were cultured in reduced medium. Cancer organoids were established from tumor specimens as donut cultures with tissue fragments seeded on top of a solidified ring of Matrigel matrix (Tan, C. W., Hirokawa, Y. & Burgess, A. W. (2015) Sci Rep 5, 11036). With this method, living intestinal epithelial cells attach to the Matrigel surface while other cell types and tissue debris accumulate at the center. Following primary organoid establishment, cultures were passaged as dome cultures with organoids embedded in a solidified drop of Matrigel matrix.

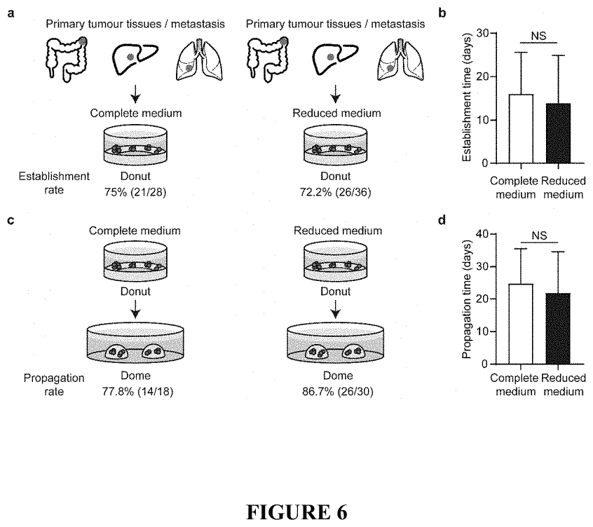

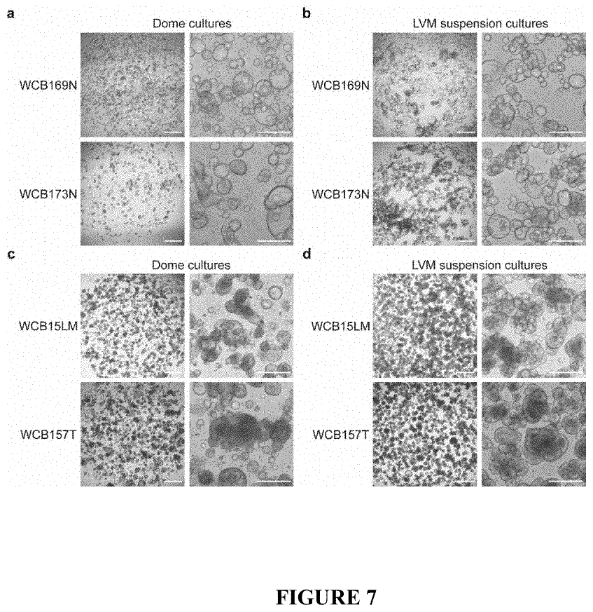

Success of cancer organoid establishment (passage 0 to 1) in donut cultures was similar for complete and reduced media, with establishment rates of 75.0% (21/28) and 72.2% (26/36; P=1.000) and mean establishment times of 16.0 days (s.d.=9.6) and 13.9 days (s.d.=11.0; P=0.491), respectively (FIGS. 6a and b and FIG. 7). For organoids propagated (passage 1 to 2) in dome culture, success was also similar with propagation rates of 77.8% (14/18) and 86.7% (26/30; P=0.451) and propagation times of 24.7 days (s.d.=10.8) and 21.8 days (s.d.=12.8; P=0.470), respectively (FIGS. 6c and d and FIG. 7).

Taking into consideration stability, quality and efficacy, for further method development the IntestiCult Organoid Growth Medium was selected for culture of normal colorectal epithelium and the reduced medium for culture of colorectal cancer tissue.

### Example 6 Development of a LVM Suspension Culture Method for Propagation of Colorectal Normal and Cancer Organoids

The use of a solid support matrix is poorly suited to organoid maintenance and experimental manipulation. To address this challenge, and based on previous findings from donut cultures that intestinal organoids can establish when only partially touching a support matrix, the inventors examined whether the traditional solid matrix could be replaced with a low-viscosity (5% Matrigel) matrix suspension which, unlike high-percentage Matrigel matrix, does not solidify at 37° C.

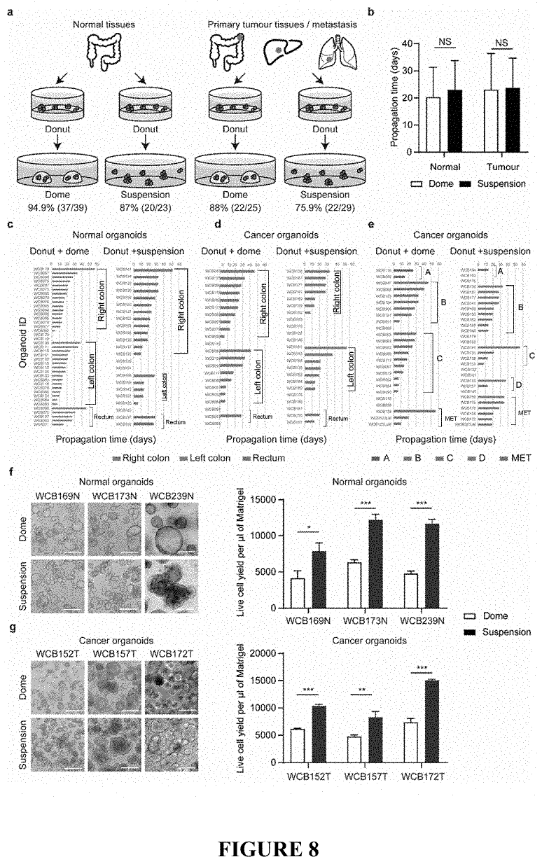

To compare LVM suspension and Matrigel dome culture methods for intestinal organoid propagation (passage 1 to 2), cultures were established for 62 normal colorectal and 54 cancer tissues. For organoids derived from normal colorectal epithelium, LVM suspension cultures achieved similar success as compared to dome cultures, with propagation rates of 87.0% (20/23) and 94.9% (37/39; P=0.350), respectively (FIG. 8a). Corresponding results were obtained for cancer organoids, with propagation rates of 75.9% (22/29) in LVM suspension and 88.0% in dome culture (22/25; P=0.310) (FIG. 8a). There was no significant difference in propagation times between organoids grown using either method for both normal organoids (LVM: mean=23.1 days, s.d.=10.6; dome: mean=20.3 days, s.d.=11.0; P=0.362) and cancer organoids (LVM: mean=21.5 days, s.d.=13.9; dome: mean=24.2 days, s.d.=13.0; P=0.854) (FIG. 8b). Propagation rates and times were similar for normal colorectal organoids irrespective of the intestinal tract location (FIG. 8c, Table 2 and 3 and for cancer organoids irrespective of location and tumor stage (FIG. 8d, e; Tables 2 and 3).

TABLE 2
Propagation rates for intestinal organoids derived from 62 normal colorectal and 54
cancer tissues grown in low-viscosity matrix suspension or Matrigel dome culture

| | | | | | | LVM | | | | |
|---|---|---|---|---|---|---|---|---|---|---|
| Propagation | | Dome culture | | | | suspension culture | | | | Dome vs |
| PDTO | | | | Success | P | | | Success | P | Suspension |
| type | Characteristic | Yes | No | rate (%) | value | Yes | No | rate (%) | value | P value |
| Normal | Site | | | | | | | | | |
| | Right | 16 | 2 | 88.9 | 0.207 | 13 | 2 | 86.7 | 1.000 | 1.000 |
| | Left/Rectum | 21 | 0 | 100.0 | | 7 | 1 | 87.5 | | 0.276 |
| Tumor | Site | | | | | | | | | |
| | Right | 10 | 1 | 90.9 | 1.000 | 7 | 4 | 63.6 | 0.667 | 0.311 |
| | Left/Rectum | 9 | 2 | 81.8 | | 9 | 3 | 75.0 | | 1.000 |
| | Stage | | | | | | | | | |
| | I-III | 19 | 3 | 86.4 | 1.000 | 14 | 4 | 77.8 | 0.277 | 0.680 |
| | IV (incl. | 3 | 0 | 100.0 | | 8 | 0 | 100.0 | | 1.000 |
| | metastasis) | | | | | | | | | |
| | Differentiation | | | | | | | | | |
| | Well/Moderate | 16 | 1 | 94.1 | 0.352 | 15 | 6 | 71.4 | 0.318 | 0.104 |
| | Poor | 3 | 1 | 75.0 | | 0 | 1 | 0 | | 0.400 |

Statistical significance was attributed to values of P<0.05 as determined by the Fisher's exact test.

TABLE 3
Propagation times for intestinal organoids derived from 62 normal colorectal and 54
cancer tissues grown in low-viscosity matrix suspension or Matrigel dome culture.

| | | | | | | LVM | | | | |
|---|---|---|---|---|---|---|---|---|---|---|
| Propagation | | Dome culture | | | | suspension culture | | | | Dome vs |
| PDTO | | | | | P | | | | P | Suspension |
| type | Characteristic | N | Mean | S.D. | value | N | Mean | S.D. | value | P value |
| Normal | Site | | | | | | | | | |
| | Right | 16 | 20.2 | 11.7 | 0.949 | 13 | 25.5 | 10.5 | 0.181 | 0.233 |
| | Left/Rectum | 21 | 20.4 | 10.2 | | 7 | 18.7 | 8.4 | | 0.702 |
| Tumor | Site | | | | | | | | | |
| | Right | 10 | 23.5 | 10.7 | 0.626 | 7 | 21.7 | 9.0 | 0.732 | 0.739 |
| | Left/Rectum | 9 | 20.8 | 11.9 | | 9 | 23.9 | 13.2 | | 0.628 |
| | Stage | | | | | | | | | |
| | I-III | 19 | 22.2 | 11.4 | 0.450 | 14 | 22.6 | 11.5 | 0.534 | 0.918 |
| | IV (incl. | 3 | 28.7 | 19.8 | | 8 | 25.8 | 8.6 | | 0.765 |
| | metastasis) | | | | | | | | | |
| | Differentiation | | | | | | | | | |
| | Well/Moderate | 16 | 20.5 | 10.6 | 0.145 | 15 | 23.9 | 11.3 | N/A | 0.407 |
| | Poor | 3 | 31.3 | 10.9 | | 0 | N/A | N/A | | N/A |

Organoids in LVM suspension culture could be readily biobanked and recovered with 100% (12/12) and 94.7% (18/19) success rate for normal and cancer organoids, respectively.

The inventors further examined whether organoids propagated in LVM suspension or dome culture produced similar yields of viable cells after a 14-day incubation period. Both normal and cancer organoids from three patients with colorectal cancer were grown from single cells (100,000 cells in 3.5 ml/well) either suspended in 5% of Matrigel matrix or embedded in solid Matrigel matrix with regular media changes every 2 days. For both normal and cancer tissues, compared to the traditional dome cultures, organoids grown in LVM suspension culture produced greater yields of viable cells (normal organoids: 1.7 to 2.6-fold increase; cancer organoids: 1.6 to 2.2-fold increase; P<0.05 for all comparisons) (FIG. 8 f, g).

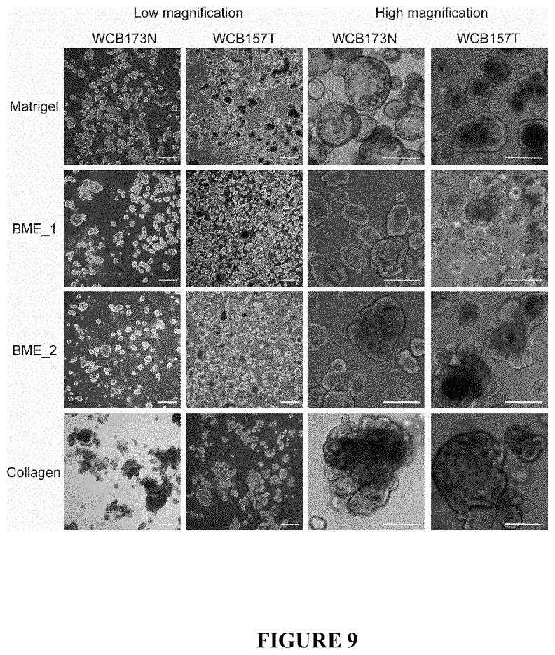

Besides Matrigel matrix, other commonly used support matrices for intestinal organoid cultures include BME-1, BME-2 and collagen type I-A (Zhang, K. & Manninen, A. (2019) Methods Mol Biol 1926, 77-84); Velagapudi, C. et al. (2012) Am J Pathol 180, 819-830). To investigate whether these alternative matrices could also support organoid growth in low-viscosity (5%) matrix culture, matched normal and cancer organoids from a representative patient were grown for 14 days as LVM suspension cultures. As observed for Matrigel, BME-1, BME-2, and collagen type I-A all supported growth of both normal and tumor organoids in LVM suspension culture (FIG. 9).

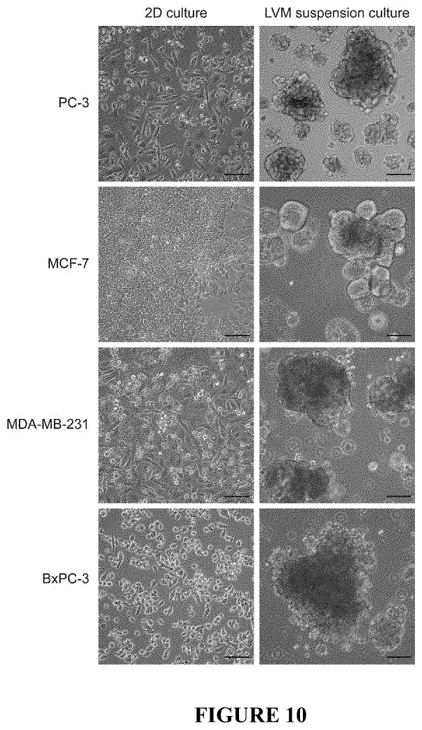

In addition, LVM suspension conditions supported the three-dimensional growth of human cancer cell lines from prostate (PC-3), breast (MDA-MB-231 and MCF-7) and pancreas (BxPC-3) cancer, suggesting that this approach may be amenable to various epithelial tissues (FIG. 10).

### Example 7 Organoid LVM Suspension Cultures Recapitulate Morphological Development Observed in Dome Cultures

Morphological development of organoids from single cells in the presence of solid support matrices has been extensively documented for normal colorectal epithelium and cancer, mirroring architectural features of the original tissue. To determine whether comparable morphological development of organoids was maintained in LVM suspension culture, representative normal and cancer cultures were monitored for growth over a 14-day period in both LVM suspension and dome culture conditions.

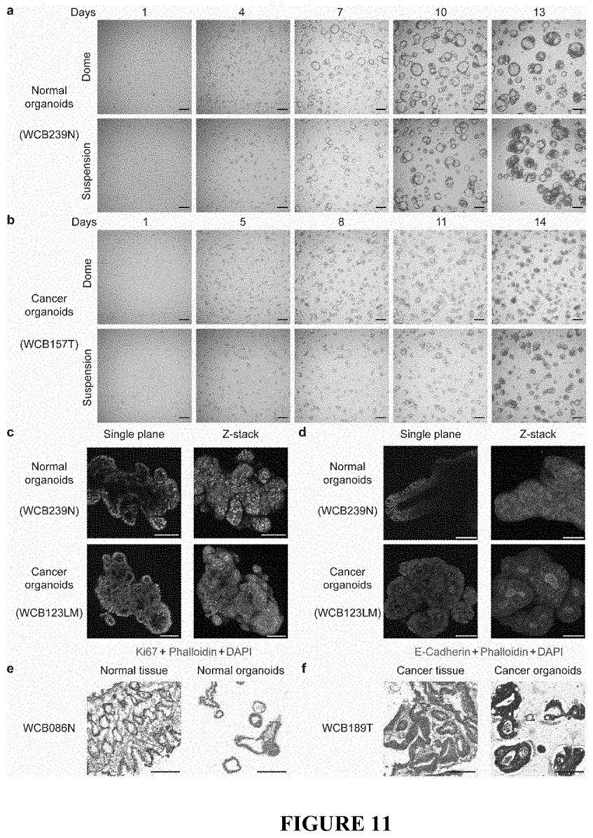

Normal colorectal organoids grown from single cells formed cystic-like structures after ˜7 days in both LVM suspension and dome culture conditions (FIG. 11a). The small normal organoids gradually ballooned out and after ˜10-13 days began to undergo budding to form crypt-like extensions. Continuous expansion of the organoids in culture for more than two weeks resulted in the formation of a large mature organoid containing numerous crypt-like features (FIG. 11c, d). Organoid structures grown in LVM suspension conditions showed structural integrity with maintenance of Ki-67 positive cells within a proliferative compartment of the crypt buds (FIG. 11c), indicating sustained growth and regular morphogenesis during long-term expansion.

For colorectal cancer organoids grown in LVM suspension or dome culture, time varied from 7 to 20 days for single cells to form heterogeneous aggregates with admixed solid and cystic morphologies (FIG. 11b). Over long-term culture in suspension, heterogeneous phenotypes and morphologies were maintained, with Ki-67 positive cells interspersed throughout organoid structures (FIG. 11c).

Expression of E-cadherin was similar between organoids grown in LVM suspension and dome culture (FIG. 11d). For both conditions, E-cadherin was expressed at cell boundaries of normal and cancer organoids.

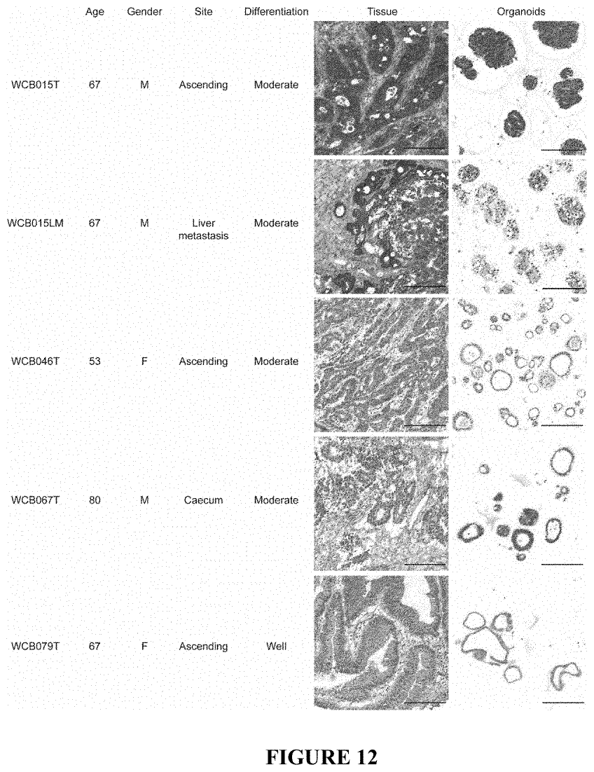

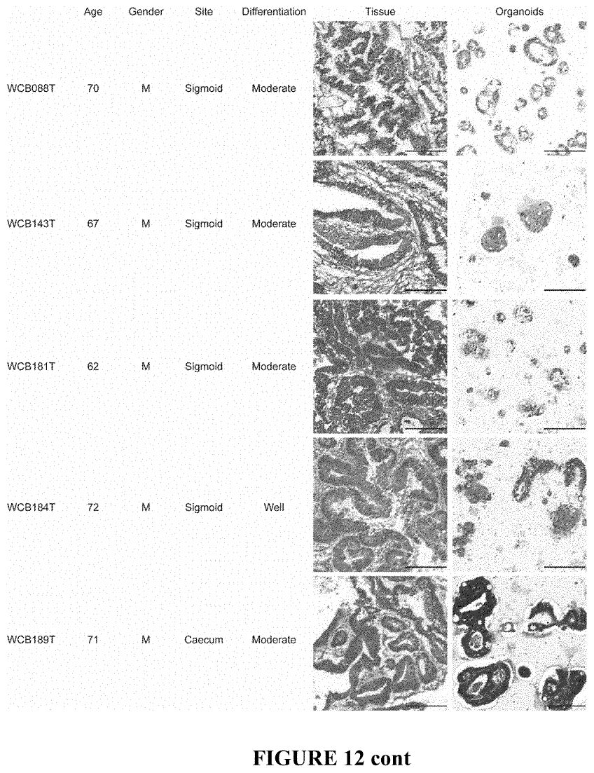

Both normal and colorectal cancer organoids grown in LVM suspension culture maintained histopathological similarities as compared to the original primary tissues (FIG. 11e, f; FIG. 12).

### Example 8 Colorectal Cancer Organoids Propagated in LVM Suspension Culture are Representative of Primary Tumors at the Genomic Level

Twenty-six colorectal cancer organoids were analyzed for mutations by whole-genome sequencing. In the absence of matched normal organoids, putative somatic mutations were identified for protein-coding exons by annotation against databases of known human germline variants as well as five normal reference samples sequenced on the same platform.

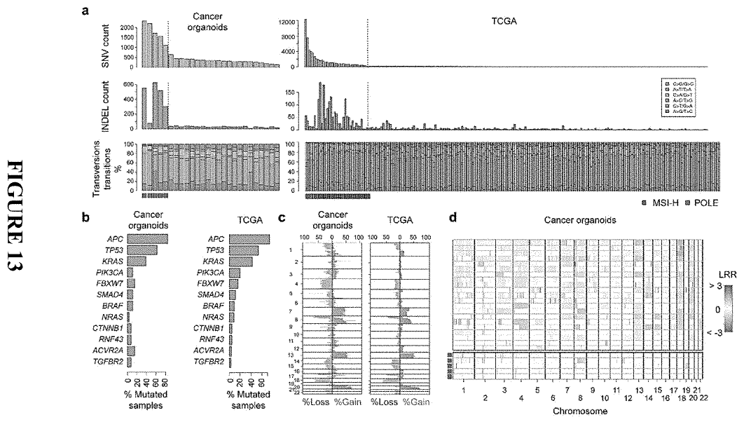

Consistent with data on primary colorectal cancers reported by the TCGA, the number of mutations varied widely among cancer organoids, ranging from 1.7 to 30.1 per 10⁶ bases (FIG. 13). Hypermutation with confirmed DNA mismatch-repair deficiency (dMMR) was 12.5% (28/224) of hypermutated cases among the TCGA cancers (P=0.357). Cancer organoids and TCGA cancers further showed similar mutation frequencies for major colorectal cancer-associated driver genes (FIG. 13b). Global DNA copy number alterations in cancer organoids mirrored those in TOGA cancers with frequent deletion of chromosome arms 8p, 17p (including TP53), and 18q (including SMAD4), and gain of chromosomes 7, 8q (including MYC), 13, and 20q (FIG. 13c). Consistent with the well-established associations in primary cancers, cancer organoids with dMMR exhibited stable DNA copy-number profiles (FIG. 13d).

### Example 9 LVM Suspension Culture Facilitates Organoid Establishment from Colorectal Normal and Cancer Tissues

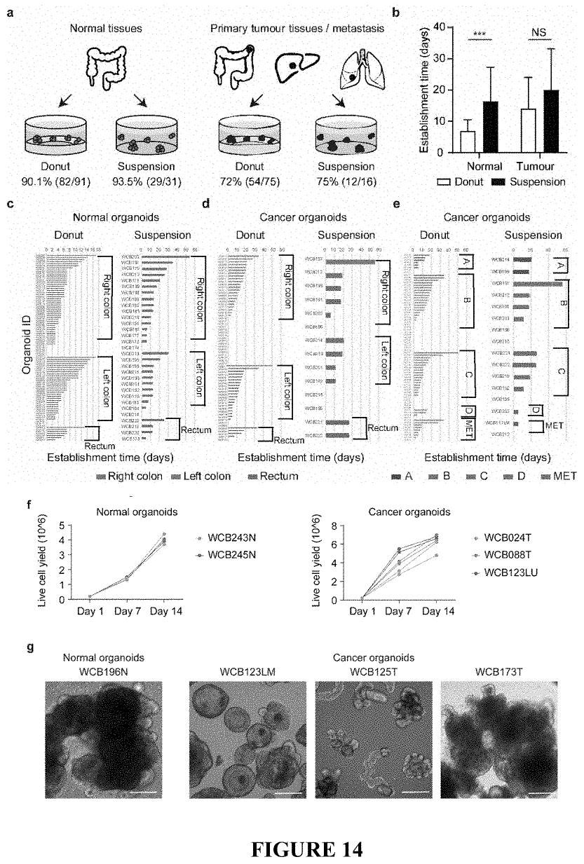

We evaluated whether LVM suspension culture could replace Matrigel donut culture for organoid establishment utilizing a consecutive series of 122 normal colorectal tissues and 91 cancer tissues. For normal colorectal tissues establishment success was similar for both methods, with establishment rates of 93.5% (29/31) for LVM suspension and 90.1% (82/91; P=0.728) for donut cultures (FIG. 14a). LVM suspension cultures sustained larger organoid numbers and sizes as compared to donut culture, allowing for an increased time to first passage with a mean of 16.4 days (s.d.=10.9) as compared to 6.9 days (s.d.=3.6; P<0.0001), respectively (FIG. 14b). Findings were similar for normal organoids derived from the right colon, left colon and rectum (FIG. 14c; Tables 4 and 5).

TABLE 4
Establishment rates for intestinal organoids derived from 122 normal colorectal tissues and
91 cancer tissues grown in low-viscosity matrix suspension or Matrigel donut culture. Statistical
significance was attributed to values of p < 0.05 as determined by the Fisher’s exact. *p < 0.05

| | | | | | | LVM | | | | |
|---|---|---|---|---|---|---|---|---|---|---|
| Establishment | | Donut culture | | | | suspension culture | | | | Donut vs |
| PDTO | | | | Success | P | | | Success | P | Suspension |
| type | Characteristic | Yes | No | rate (%) | value | Yes | No | rate (%) | value | P value |
| Normal | Site | | | | | | | | | |
| | Right | 44 | 6 | 88.0 | 0.506 | 15 | 1 | 93.8 | 1.000 | 1.000 |
| | Left/Rectum | 38 | 3 | 92.7 | | 14 | 1 | 93.3 | | 1.000 |
| Tumor | Site | | | | | | | | | |
| | Right | 22 | 17 | 56.4 | 0.006* | 5 | 1 | 83.3 | 1.000 | 0.377 |
| | Left/Rectum | 24 | 3 | 88.9 | | 6 | 2 | 75.0 | | 0.568 |
| | Stage | | | | | | | | | |
| | I-III | 43 | 19 | 69.4 | 0.330 | 10 | 3 | 76.9 | 1.000 | 0.744 |
| | IV (incl. | 11 | 2 | 84.6 | | 2 | 1 | 66.7 | | 0.489 |
| | metastasis) | | | | | | | | | |
| | Differentiation | | | | | | | | | |
| | Well/Moderate | 40 | 16 | 71.4 | 0.440 | 10 | 3 | 76.9 | 1.000 | 1.000 |
| | Poor | 5 | 4 | 55.6 | | 1 | 0 | 100 | | 1.000 |

TABLE 5
Supplementary Table 4. Establishment times for intestinal organoids derived from 122 normal
colorectal tissues and 91 cancer tissues grown in low-viscosity matrix suspension or Matrigel donut culture.
Statistical significance was attributed to values of p < 0.05 as determined by the Student’s t test. *p < 0.05

| Establishment | | Donut culture | | | | LVM | | | | Donut vs |
|---|---|---|---|---|---|---|---|---|---|---|
| PDTO | | | Mean | | | suspension culture | | | | Suspension |
| type | Characteristic | N | (days) | S.D. | P value | N | Mean | S.D. | P value | P value |
| Normal | Site | | | | | | | | | |
| | Right | 44 | 6.2 | 3.2 | 0.059 | 15 | 18.6 | 12.8 | 0.270 | <0.001* |
| | Left/Rectum | 38 | 7.7 | 3.9 | | 14 | 14.1 | 7.1 | | <0.001* |
| Tumor | Site | | | | | | | | | |
| | Right | 22 | 14.6 | 7.8 | 0.871 | 5 | 23.4 | 17.0 | 0.646 | 0.106 |
| | Left/Rectum | 24 | 14.2 | 10.9 | | 6 | 19.5 | 6.0 | | 0.272 |
| | Stage | | | | | | | | | |
| | I-III | 43 | 14.6 | 9.7 | 0.452 | 10 | 22.8 | 12.0 | 0.101 | 0.030* |
| | IV (incl. | 11 | 12.1 | 9.7 | | 2 | 6.0 | 0.0 | | 0.432 |
| | metastasis) | | | | | | | | | |
| | Differentiation | | | | | | | | | |
| | Well/Moderate | 40 | 13.7 | 9.2 | 0.216 | 10 | 22.8 | 12.0 | N/A | 0.013* |
| | Poor | 5 | 19.4 | 11.5 | | 1 | 6.0 | 0.0 | | N/A |

For cancer organoids, establishment success was also similar for LVM suspension and donut culture methods, with establishment rates of 75.0% (54/75) and 72.0% (12/16, P=1.000), respectively (FIG. 14a). As observed for normal tissues, time to first passage for cancer organoids could be prolonged in LVM suspension culture (mean=20.0 days; s.d.=13.2) as compared to donut culture (mean=14.1 days; s.d.=9.9 days), although this difference was not statistically significant (P=0.084) (FIG. 14b). Similar patterns were observed irrespective of location or tumor stage (FIG. 14d, e and Tables 4 and 5) (

### Example 10 Scalable Expansion of Intestinal Normal and Cancer Organoids in Bioreactor Tubes

A limitation of current organoid culture techniques that require a solid support matrix is scalable organoid expansion. To examine whether the LVM suspension conditions were amenable to bioreactor applications, independent patient-derived normal (n=2) and cancer (n=3) organoids, were suspended as single cells (200,000 cells) in 50 ml Bioreactor Tubes containing 7 ml of medium with 5% Matrigel. Organoids were cultured in duplicate with media changes (50:50) and organoid agitation by pipetting every 2-5 days.

Bioreactor cultures of normal and cancer organoids maintained morphological features similar to LVM suspension cultures in 6-well plates over a 14-day period (FIG. 14g), and demonstrated substantial increase in yield of viable cells achieving 3.70×10⁶ to 4.05×10⁶ cells for normal organoids and 4.80×10⁶ to 7.00×10⁶ cells for cancer organoids (FIG. 14f). Continuous expansion of organoids in bioreactor tubes for 4-8 weeks resulted in progressive enlargement of organoids and/or organoid aggregates. For normal organoids, long-term bioreactor culture generated organoids with hundreds of crypt-like protrusions, while for colorectal cancer organoids complex heterogeneous morphologies were obtained (FIG. 14g). This bioreactor method therefore enables substantial organoid expansion within a low passage number, necessary for maintenance of organoid genomic fidelity and scalable production.

### Example 11 LVM Suspension Culture of Intestinal Organoids in 384-Well Format

To evaluate the utility of LVM suspension cultures for assay miniaturization, the inventors determined whether patient-derived colorectal cancer cells could be seeded and robustly cultured as organoid structures in a 384-well format. To facilitate automated dispensing of media with the LVM cell suspension, the Matrigel concentration was reduced to 3%; a seeding density of 3000 cells/well (in 60 μl media) was found to produce ˜150 to 300 distinct organoids suitable for imaging.

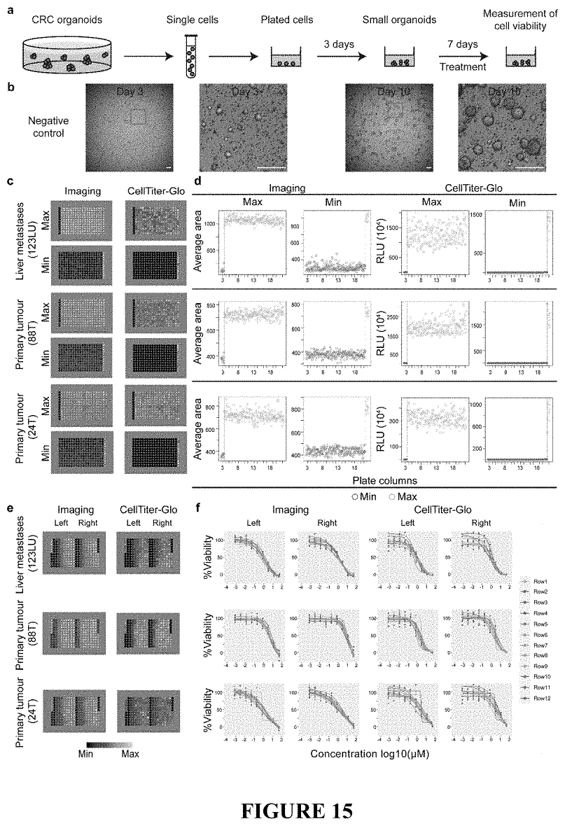

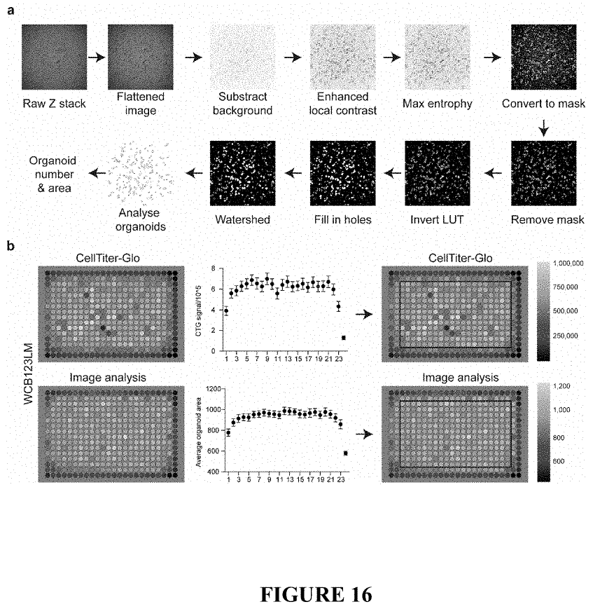

To examine cancer organoid growth in 384-well format, single-cell suspensions from three representative tumors were dispensed using a Mantis Liquid Handler. Plates were imaged every 24 hours for 10 days on an automated Eclipse Ti2 Inverted Microscope System with an integrated tissue culture incubator to measure organoid size followed by determination of cell viability (ATP consumption) using CellTitreGlo 3D reagent (FIG. 15a; and FIG. 16a). As found for larger plate formats, organoids formed within 3 days and expanded over the subsequent 7-day period without requiring media change (FIG. 15b). Due to media evaporation over the 10 day assay period, an edge effect was observed with reduced organoid growth in the outer two rows (A & B and O & P) and columns (1 & 2 and 23 & 24) (FIG. 16b)); in subsequent assays these outer wells were omitted from analysis.

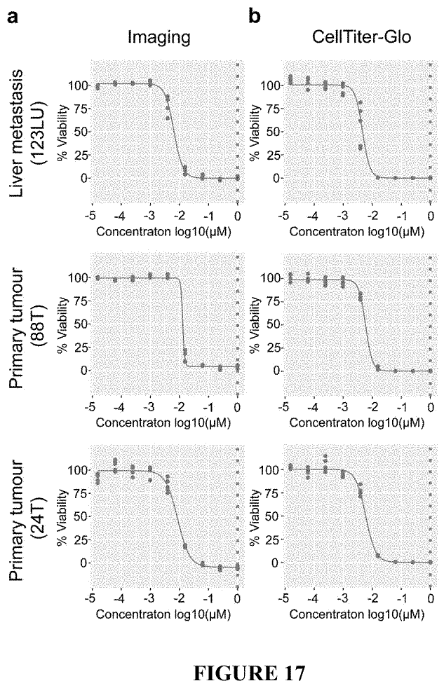

Plate uniformity in the 384-well format was evaluated for maximum (Max) signals, minimum (Min) signals, and drug dose-dependent midpoint (Mid) signals (FIG. 15c-f). To determine Max signals, organoids were established for 3 days, treated with 0.5% DMSO (vehicle control) using a JANUS Automated Liquid Handling Workstation with a 384 pin tool, and incubated for 7 days. Informed by the inventors previous studies on colorectal cancer cell lines, Min signals were determined using the proteasome inhibitor bortezomib as a cell killing control with complete growth inhibition observed at 1 μM (FIG. 17)( ). To evaluate dose-dependent Mid signals, regorafenib was titrated across each half of the assay plate in a 9-step, 4-fold dilution series starting from 50 μM, generating a total of 24 drug dose-response curves. Organoid viability was determined by both image analysis and CellTiter-Glo 3D luminescence measurements. Mean organoid size and luminescence signals were visualized as heatmaps for each plate and plotted by wells for each plate row from left to right or as drug dose-response curves.

Performance metrics for plate uniformity were compared between image analysis and CellTitre-Glo 3D assays. Image analysis produced CVs of 1.9% to 5.9% for Max signals, 2.2% to 15.4% for Min signals and 4.4% to 9.8% for Mid signals, well below the required compliance threshold of 20% for high-throughput cell-based screens (Table 6) (Iversen, P. W. et al. in Assay Guidance Manual. (eds. S. Markossian et al.) (Bethesda (Md.); 2004). Similarly, all signal windows were greater than 2 (range 4.3 to 29.0), and all robust Z′ factors were greater than 0.50 (range 0.50 to 0.82), indicating an excellent quality of the organoid assays for imaging. In contrast, the inventors observed an overall poorer performance for CellTiter-Glo assays (Table 7). (CVs ranged from 8.2% to 24.0% for Max signals, 13.8% to 89.2% for Min signals and 18.0% to 29.3% for Mid signals. Some signal windows were less than 2 (range 1.13 to 9.02), and some robust Z′ factors were less than 0.50 (range 0.16 to 0.77).

TABLE 6
Performance metrics for plate uniformity for cancer organoids assessed using image
analysis. Plate uniformity in 384-well format was evaluated for three different cancer organoids for
maximum (Max) signals, minimum (Min) signals, and drug dose-dependent midpoint (Mid) signals based
on organoid size measurements with the latter reported for each side of each plate (left and right). CV,
coefficient of variation; S.D. standard deviation; SW, signal window; Z′, Z′ factor. N = 228 for Max (Max
plates) and Min (Min plates). N = 12 for Max (Min plates), Min (Max plates), and Mid (Mid plates, left and
right). N = 6 for Max (Mid plates left and right), Min (Mid plates, left and right).
Imaging

| Organoid | | | Mean | | | | Z′ | Mean ED₅₀ | Mean ED₅₀ |
|---|---|---|---|---|---|---|---|---|---|
| ID | Plate | Type | (size) | S.D. | CV | SW | (robust Z′) | (μM) | Fold Change |
| WCB024T | Mid (Left) | Max | 942 | 38 | 4.0 | 10.9 | 0.73 (0.82) | 1.54 | 1.5 |
| | | Min | 376 | 13 | 3.5 | | | | |
| | | 0.78 uM | 699 | 69 | 9.8 | | | | |
| | Mid (Right) | Max | 963 | 37 | 3.8 | 13.1 | 0.78 (0.73) | 2.28 | |
| | | Min | 353 | 8 | 2.2 | | | | |
| | | 0.78 uM | 730 | 53 | 7.3 | | | | |
| | Max | Max | 714 | 42 | 5.9 | 4.3 | 0.51 (0.55) | | |
| | | Min | 361 | 15 | 4.1 | | | | |
| | Min | Max | 809 | 39 | 4.8 | 4.7 | 0.48 (0.50) | | |
| | | Min | 427 | 35 | 6.1 | | | | |
| WCB088T | Mid (Left) | Max | 623 | 22 | 3.5 | 8.2 | 0.59 (0.60) | 6.09 | 1.7 |
| | | Min | 325 | 19 | 5.8 | | | | |
| | | 12.50 uM | 395 | 17 | 4.4 | | | | |
| | Mid (Right) | Max | 617 | 25 | 4.1 | 7.1 | 0.61 (0.80) | 10.15 | |
| | | Min | 326 | 13 | 4.1 | | | | |
| | | 12.50 uM | 441 | 27 | 6.2 | | | | |
| | Max | Max | 722 | 30 | 4.1 | 6.4 | 0.55 (0.60) | | |
| | | Min | 378 | 22 | 5.8 | | | | |
| | Min | Max | 736 | 31 | 4.3 | 6.1 | 0.54 (0.64) | | |
| | | Min | 378 | 25 | 6.1 | | | | |
| WCB123LU | Mid (Left) | Max | 934 | 45 | 4.8 | 7.5 | 0.55 (0.68) | 1.23 | 1.3 |
| | | Min | 315 | 49 | 15.4 | | | | |
| | | 0.78 uM | 661 | 35 | 5.3 | | | | |
| | Mid (Right) | Max | 900 | 17 | 1.9 | 29.0 | 0.82 (0.82) | 1.56 | |
| | | Min | 286 | 18 | 6.5 | | | | |
| | | 0.78 uM | 673 | 32 | 4.7 | | | | |
| | Max | Max | 1052 | 46 | 4.4 | 10.8 | 0.67 (0.75) | | |
| | | Min | 303 | 37 | 12.3 | | | | |
| | Min | Max | 898 | 41 | 4.5 | 8.8 | 0.61 (0.60) | | |
| | | Min | 302 | 27 | 12.2 | | | | |

TABLE 7
Performance metrics for plate uniformity for cancer organoids assessed using CellTitre-Glo
3 D assays. Plate uniformity in 384-well format was evaluated for three different cancer organoids for
maximum (Max) signals, minimum (Min) signals, and drug dose-dependent midpoint (Mid) signals based
on fluorescence measurements with the latter reported for each side of each plate (left and right). CV,
coefficient of variation; S.D. standard deviation; SW, signal window; Z′, Z′ factor. N = 228 for Max (Max
plates) and Min (Min plates). N = 12 for Max (Min plates), Min (Max plates), and Mid (Mid plates, left and
right). N = 6 for Max (Mid plates left and right), Min (Mid plates, left and right).
CellTitre-Glo 3 D

| | | | | | | | | Mean | Mean |
|---|---|---|---|---|---|---|---|---|---|
| Organoid | | | Mean | | | | Z′ | ED₅₀ | ED₅₀ Fold |
| ID | Plate | Type | (intensity) | S.D. | CV | SW | (robust Z′) | (μM) | Change |
| WCB024T | Mid (Left) | Max | 695975 | 100078 | 14.4 | 3.9 | 0.56 (0.71) | 3.69 | 1.4 |
| | | Min | 2323 | 1130 | 48.7 | | | | |
| | | 3.13 μM | 350277 | 85107 | 24.3 | | | | |
| | Mid (Right) | Max | 863768 | 123802 | 14.3 | 3.9 | 0.56 (0.73) | 5.15 | |
| | | Min | 7667 | 2244 | 29.3 | | | | |
| | | 3.13 μM | 424756 | 82633 | 19.5 | | | | |
| | Max | Max | 323298 | 47655 | 14.7 | 3.7 | 0.55 (0.51) | | |
| | | Min | 2730 | 376 | 13.8 | | | | |
| | Min | Max | 860127 | 169492 | 19.7 | 2.1 | 0.41 (0.40) | | |
| | | Min | 860127 | 169492 | 37.0 | | | | |
| WCB088T | Mid (Left) | Max | 1408517 | 256294 | 18.2 | 2.5 | 0.45 (0.35) | 1.98 | 1.7 |
| | | Min | 1908 | 1468 | 76.9 | | | | |
| | | 3.13 μM | 544057 | 159171 | 29.3 | | | | |
| | Mid (Right) | Max | 1516397 | 124227 | 8.2 | 9.0 | 0.75 (0.76) | 3.41 | |
| | | Min | 13263 | 3172 | 23.9 | | | | |
| | | 3.13 μM | 873049 | 183175 | 21.0 | | | | |
| | Max | Max | 1921444 | 341406 | 17.8 | 2.6 | 0.46 (0.44) | | |
| | | Min | 13318 | 2439 | 18.3 | | | | |
| | Min | Max | 1797946 | 320976 | 17.9 | 2.6 | 0.46 (0.39) | | |
| | | Min | 458 | 84 | 29.2 | | | | |
| WCB123LU | Mid (Left) | Max | 735969 | 161089 | 21.9 | 1.5 | 0.34 (0.71) | 1.01 | 1.1 |
| | | Min | 1040 | 927 | 89.2 | | | | |
| | | 0.78 μM | 434300 | 78260 | 18.0 | | | | |
| | Mid (Right) | Max | 809775 | 115061 | 14.2 | 3.9 | 0.56 (0.60) | 1.10 | |
| | | Min | 8079 | 2339 | 29.0 | | | | |
| | | 0.78 μM | 475470 | 125155 | 26.3 | | | | |
| | Max | Max | 1253442 | 300265 | 24.0 | 1.1 | 0.27 (0.16) | | |
| | | Min | 8687 | 1478 | 17.0 | | | | |
| | Min | Max | 1299146 | 198646 | 15.3 | 3.5 | 0.54 (0.77) | | |
| | | Min | 340 | 62 | 27.2 | | | | |

For both image analysis and CellTitre-Glo 3D assays, the ED₅₀ mean fold-changes between plates were all acceptable at less than two-fold (imaging: range 1.26 to 1.67; CellTitre-Glo 3D: range 1.09 to 1.73) (Iversen, P. W., Eastwood, B. J., Sittampalam, G. S. & Cox, K. L. (2006) J Biomol Screen 11, 247-252).

However, ED₅₀ values diverged (>2 fold) between image analysis and CellTitre-Glo 3D assays for two out of three organoids with higher potency estimates for viability measurements based on ATP consumption. Differential drug potency estimates for imaging and nonspecific metabolic activity assays are well-documented, and accordingly regorafenib has been shown to impair mitochondrial function and decrease cellular ATP levels (Weng, Z. et al. (2015) Toxicology 327, 10-21).

### Example 12 Utility of LVM Suspension Culture for Imaging-Based High-Throughput Drug Screening

To demonstrate the utility of LVM suspension culture for high-throughput screening applications, we evaluated the reproducibility of imaging-based drug sensitivity testing of cancer organoids for clinically relevant agents including 5-FU (pyrimidine analogue), oxaliplatin (DNA intercalating agent), SN-38 (active metabolite of the topoisomerase I inhibitor irinotecan), regorafenib (multikinase inhibitor) and TAS-102 (thymidine-based nucleic acid analogue and a thymidine phosphorylase inhibitor). Organoids were grown over the course of 3 days, followed by automated addition of drug dilutions from pre-prepared master compound plates. Drug dilutions covered the physiological concentrations of 5-FU (Cmax≈7.5 μM), oxaliplatin (Cmax≈5.0 μM), SN-38 (Cmax≈0.14 μM), regorafenib (Cmax≈7.3 μM) (NCT01853046) and TAS-102 (Cmax≈18.2 μM) (AusPAR 2018) observed in patients. All drugs were assayed in duplicate in a 9-step, 4-fold dilution series with daily imaging for 7 days. 0.5% DMSO and bortezomib (1 μM) served as negative (vehicle) and positive (PDTO killing) controls, respectively. Drug responses were evaluated using growth rate adjusted (GR) measurements based on comparing growth rates in the presence and absence of drug. Parametrization of GR data yields GR₅₀, GRmax and GRAOC values that are largely independent of division rate and assay duration (Hafner, M., Niepel, M., Chung, M. & Sorger, P. K. (2016) Nat Methods 13, 521-527).

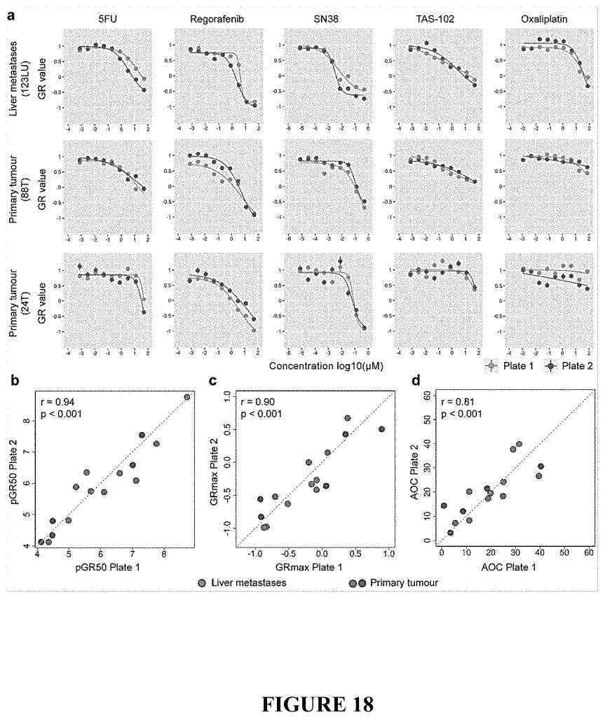

Two independent runs were performed for three colorectal cancer organoids. Four-parameter logistic regression was used to fit drug dose-response curves, with high concordance of curve fits evident between runs for all five drugs (FIG. 18). Accordingly, replicate experiments showed strong correlations for pGR₅₀, GRmax and GRAOC estimates, with Pearson's correlation coefficients of 0.94, 0.90 and 0.81, respectively (p<0.001 for all comparisons) (FIG. 18b-d).

### REMARKS

The data provided herein shows that solid support matrices can be replaced with a LVM suspension preparation to enable establishment, propagation and expansion of normal and cancer intestinal organoids. Organoids grown in LVM suspension recapitulate the morphological development of organoids produced in solid matrix cultures. The LVM derived cancer organoids reflect the histological and genetic heterogeneity of primary colorectal cancers.

Furthermore, it has been demonstrated that LVM suspension culture enables rapid scalable expansion of organoids in Bioreactor tubes required to produce sufficient cells from high throughput assay applications.

1.-26. (canceled)

27. A method of producing and propagating organoids, the method comprising:
(i) providing a stem cell culture medium comprising low viscosity extracellular matrix and a cell culture support medium;
(ii) seeding the stem cell culture medium with stem cells; and
(iii) establishing a stem cell culture in the stem cell culture medium under a time and conditions sufficient for the stem cells to generate organoids; and
(iv) propagating the organoids in the stem cell culture medium.

28. The method according to claim 27, comprising periodically agitating the stem cell culture and/or the propagating organoids in the stem cell culture medium.

29. The method according to claim 28 wherein the periodical agitation is performed during the establishing step and/or during the propagating step.

30. The method according to claim 27, wherein the stem cells are cultured in suspension culture.

31. The method according to claim 27, wherein the low viscosity extracellular matrix is a basement membrane protein mixture extracted from Engelbreth-Holm-Swarm (EHS) mouse sarcoma.

32. The method according to claim 27, wherein the low viscosity extracellular matrix remains in liquid form at a temperature between about 35° C. and 39° C.

33. The method according to claim 27, wherein the low viscosity extracellular matrix is 3-5% of the culture media volume.

34. The method according to claim 27, wherein the low viscosity extracellular matrix comprises laminin, collagen IV, enactin and proteoglycans.

35. The method according to claim 27, wherein the low viscosity extracellular matrix is supplemented with one or more growth factors.

36. The method according to claim 27, wherein the cell culture support medium is a liquid basal medium supplemented with one or more antibiotics and/or anti-fungal agents.

37. The method according to claim 36, wherein the liquid basal medium is supplemented with one or more of nicotinamide, N-acetyl-L-cysteine, B27 supplement, N2 supplement and HEPES buffer and optionally heparin.

38. The method according to claim 36, wherein the cell culture support medium further comprises a ROCK1 inhibitor.

39. The method according to claim 27, wherein the stem cells are embryonic stem cells (ESC), mesenchymal stem cells (MSC), induced pluripotent stem cells (iPSC) or adult stem cells.

40. The method according to claim 27, wherein the method provides for organoids derived from primary tumours from small intestine, colon, rectum, skin, breast, oesophagus, head and neck, liver, lung, prostate, kidney, bladder, thyroid, endometrial, stomach and pancreas.

41. The method according to claim 27, wherein the method provides for organoids derived from metastatic tumours from small intestine, colon, rectum, skin, breast, oesophagus, head and neck, liver, lung, prostate, kidney, bladder, thyroid, endometrial, stomach and pancreas.

42. The method according to claim 27 wherein the stem cell culture is established between 2 and 70 days, between 2 and 50 days, or between 2 and 20 days.

43. The method according to claim 27 wherein the generated organoids are propagated at least 14 days or at least 30 days.

44. Use of normal or tumour organoids produced by the method according to claim 1 in one or more of the following:
(i) a drug discovery screen;
(ii) large-scale organoid production;
(iii) functional assays;
(iv) genetic testing assays;
(v) drug testing assays;
(vi) infection assays;
(vii) pathogen expansion assays,
(viii) co-culture assays;
(ix) toxicity assays; and
(x) regenerative medicine.

45. A population of organoids obtained according to the method of claim 27.

46. A population of organoids obtained according to the method of claim 40.
